(19) United States
(12) Patent Application Publication
(10) Pub. No.: US 2009/0325859 A1
(43) Pub. Date: Dec. 31, 2009

(54) CITRIC ACID POLYMERS

(75) Inventors: Guillermo Ameer, Chicago, IL (US); Jian Yang, Arlington, TX (US); Ryan Hoshi, Chicago, IL (US)

Correspondence Address: Casimir Jones, S.C., 2275 DEMING WAY, SUITE 310, MIDDLETON, WI, 53562, US

(73) Assignee: NORTHWESTERN UNIVERSITY, Evanston, IL (US)

(21) Appl. No.: 12/488,306

(22) Filed: Jun. 19, 2009

Related U.S. Application Data
(63) Continuation-in-part of application No. 12/370,312, filed on Feb. 12, 2009.
Application No. 12/370,312 is a division of application No. 10/945,354, filed on Sep. 20, 2004, now abandoned.
(60) Provisional application No. 60/503,943, filed on Sep. 19, 2003.
(60) Provisional application No. 60/556,642, filed on Mar. 26, 2004.
(60) Provisional application No. 61/074,348, filed on Jun. 20, 2008.

(51) Int. Cl. A61K 47/48; A61K 47/34; A61K 48/00; A61K 38/02; C08G 63/12; C08L 65/00

(52) U.S. Cl. 514/2; 514/772.3; 514/44 R; 528/296; 524/600

(57) ABSTRACT

The present invention provides polymers (e.g., elastomeric citric acid polymers) and methods of making and using these polymers (e.g., as a biologically active molecule delivery platform). In certain embodiments, the polymer has adsorbed biologically active molecules. In particular embodiments, the polymer comprises pores that are between about 7 and 15 nanometers in diameter. In other embodiments, the polymer comprises poly(1,8 octanediol-co-ctric acid). In certain embodiments, the polymers are made by employing polyethylene glycol dimethyl ether (PEGDM).

### CROSS REFERENCE TO RELATED APPLICATIONS

The present application is a continuation-in-part of U.S. patent application Ser. No. 12/370,312, filed Feb. 12, 2009, which is a divisional of U.S. patent application Ser. No. 10/945,354, filed Sep. 20, 2004, which claims priority to U.S. Provisional Patent Application Ser. No. 60/503,943 filed Sep. 19, 2003, and U.S. Provisional Patent Application Ser. No. 60/556,642, filed Mar. 26, 2004, all of which are herein incorporated by reference. The present application also claims priority to U.S. Provisional Patent Application Ser. No. 61/074,348 filed Jun. 20, 2008, which is herein incorporated by reference.

### GOVERNMENT SUPPORT

This invention was made with government support under Grant No. R21HL71921-02 awarded by the National Institutes of Health. The government has certain rights in the invention.

### FIELD OF THE INVENTION

The present invention relates to citric acid polymers (e.g., elastomeric citric acid polyester polymers) and methods of making and using these citric acid polymers (e.g., as a biologically active molecule delivery platform). In certain embodiments, the citric acid polymer has adsorbed biologically active molecules. In particular embodiments, the citric acid polymer comprises pores that are between about 7 and 15 nanometers in diameter. In other embodiments, the citric acid polymer comprises poly(1,8 octanediol-co-ctric acid). In certain embodiments, the polymers are made by employing polyethylene glycol dimethyl ether (PEGDM).

### BACKGROUND

Controlled delivery of bioactive molecules, protein based therapeutics and other drugs for biomedical applications remain a challenge. During delivery it can be difficult to maintain drug activity and tailor drug delivery rates at the therapeutic site. In protein-based therapeutics, such as growth factors used in tissue engineering applications, proteins contain secondary, tertiary and sometimes quaternary structure that must be maintained for biological activity [51]. Several protein based therapies rapidly lose biological activity in vivo (Wang et al., 2004) and additional complications may arise if the protein becomes denatured or aggregated upon delivery [52-53].

### SUMMARY OF THE INVENTION

The present invention provides citric acid polymers (e.g., elastomeric citric acid polymers) and methods of making and using these citric acid polymers (e.g., as a biologically active molecule delivery platform). In certain embodiments, the citric acid polymer has adsorbed biologically active molecules. In particular embodiments, the citric acid polymer comprises pores that are between about 7 and 15 nanometers in diameter. In other embodiments, the citric acid polymer comprises poly(1,8 octanediol-co-ctric acid). In certain embodiments, the citric acid polymers are made by employing polyethylene glycol dimethyl ether (PEGDM).

In some embodiments, the present provides compositions comprising an elastomeric citric acid polymer (e.g., nanoporous poly(diol) citrate polymer) and a plurality of biologically active molecules, where in the nanoporous poly(diol) citrate polymer has the formula:

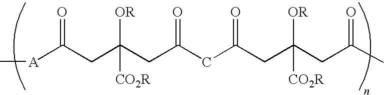

where A is a linear aliphatic dihydroxy monomer (e.g., linear aliphatic diol), C is a linear aliphatic dihydroxy monomer (e.g., linear aliphatic diol), R is hydrogen or a polymer (e.g., per the formula above, such that there is cross-linking), and n is an integer greater than 1, and wherein the plurality of biologically active molecules are adsorbed to the nanoporous poly(diol) citrate polymer. In certain embodiments, the polymer is an elastomer.

In certain embodiments, the present invention provides compositions comprising an elastomeric citric acid polymer (e.g., nanoporous poly(diol) citrate polymer) having the formula:

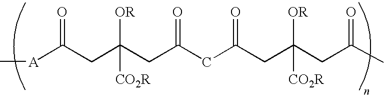

, where A is a linear aliphatic dihydroxy monomer (e.g. linear aliphatic diol), C is a linear aliphatic dihydroxy monomer (e.g., linear aliphatic diol), R is hydrogen or a polymer (e.g., per the formula above, such that there is cross-linking), and n is an integer greater than 1. In further embodiments, the nanoporous poly(diol) citrate polymer comprises pores between about 7 and about 15 nanometers. In some embodiments, the compound is an elastomer.

In particular embodiments, the present invention provides compositions comprising an elastomeric citric acid polymer (e.g., a nanoporous poly(diol) citrate polymer) and at least trace amounts of a non-reactive porogen, wherein the nanoporous poly(diol) citrate polymer has the formula

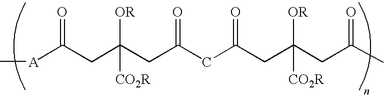

, where A is a linear aliphatic dihydroxy monomer (e.g., linear aliphatic diol), C is a linear aliphatic dihydroxy monomer (e.g., linear aliphatic diol), R is hydrogen or a polymer (e.g., per the formula above, such that there is cross-linking) and n is an integer greater than 1. In particular embodiments, the nonreactive porogen comprises polyethylene glycol dimethyl ether (PEGDM). In some embodiments, the compound is an elastomer.

In certain embodiments, A is a linear aliphatic diol comprising between about 2 and about 20 carbons. In some embodiments, C is a linear diol comprising between about 2 and about 20 carbons. In other embodiments, both A and C are the same linear diol. In further embodiments, the nanoporous poly(diol) citrate polymer is biodegradable. In other embodiments, the the nanoporous poly(diol) citrate polymer comprises pores between about 7 and about 15 nanometers (e.g., 7, 8, 9, 10, 11, 12, 13, 14, or 15). In some embodiments, the nanoporous poly(diol) citrate polymer comprises poly(1,8 octanediol-co-ctric acid). In additional embodiments, the compositions further comprise at least a trace amount of a non-reactive porogen, wherein the non-reactive porogen is impregnated in the nanoporous poly(diol) citrate polymer. In certain embodiments, the compositions further comprise at least a trace amount of polyethylene glycol dimethyl ether (PEGDM), wherein the PEGDM is impregnated in the nanoporous poly(diol) citrate polymer.

In other embodiments, the compositions further comprise a plurality of biologically active molecules that are absorbed to the nanoporous poly(diol) citrate polymer. In some embodiments, the plurality of biologically active molecules are selected from the group consisting of: proteins, nucleic acids, drugs, pro-drugs, and small molecules. In further embodiments, the plurality of biologically active molecules are selected from the group consisting of: proteins, nucleic acids, drugs, pro-drugs, and small molecules.

In some embodiments, the present invention provides methods of making a nanoporous poly(diol) citrate polymer comprising: a) combining a plurality of diol molecules, citric acid, and a plurality of non-reactive porogen molecules, under conditions such that an intermediate composition is generated, and b) contacting the intermediate composition with water or an organic solvent under conditions such that all or most of the non-reactive porogen molecules are leached out of the intermediate composition, thereby generating a nanoporous poly(diol) citrate polymer having the formula (A−B−C−B)ₙ, where A is a linear aliphatic diol, B is citric acid, C is a linear aliphatic diol, and n is an integer greater than 1.

In certain embodiments, the intermediate composition comprises about 50-75% of the non-reactive porogen molecules. In other embodiments, the non-reactive porogen molecules comprise polyethylene glycol dimethyl ether (PEGDM). In some embodiments, the nanoporous poly(diol) citrate polymer comprises pores between about 7 and about 15 nanometers.

In particular embodiments, the present invention provides methods of treating a patient comprising: administering one of the compounds described above or below.

In other embodiments, the present invention provides compositions comprising a nanoporous poly(diol) citrate polymer having the formula (A−B−C−B)ₙ, where A is a linear aliphatic diol, B is citric acid, C is a linear aliphatic diol, and n is an integer greater than 1, and the nanoporous poly(diol) citrate polymer comprises pores between about 7 and about 15 nanometers.

In certain embodiments, the present invention provides methods for making a nanoporous poly(diol) citrate polymer comprising: a) combining a plurality of diol molecules, citric acid, and a plurality of non-reactive porogen molecules, under conditions such that an intermediate composition is generated, and b) contacting the intermediate composition with water or an organic solvent under conditions such that all or most of the non-reactive porogen molecules are leached out of the intermediate composition, thereby generating a nanoporous poly(diol) citrate polymer having the formula (A−B−C−B)ₙ, where A is a linear aliphatic diol, B is citric acid, C is a linear aliphatic diol, and n is an integer greater than 1.

In further embodiments, the present invention provides compositions comprising a nanoporous poly(diol) citrate polymer and at least trace amounts of a non-reactive porogen, wherein the nanoporous poly(diol) citrate polymer has the formula (A−B−C−B)ₙ, where A is a linear aliphatic diol, B is citric acid, C is a linear aliphatic diol, and n is an integer greater than 1.

In some embodiments, the nanoporous poly(diol) citrate polymer is biodegradable. In particular embodiments, the nanoporous poly(diol) citrate polymer comprises pores between about 7 and about 15 nanometers (e.g., 7, 8, 9, 10, 11, 12, 13, 14, or 15). In further embodiments, the nanoporous poly(diol) citrate polymer comprises poly(1,8 octanediol-co-ctric acid) (aka “POC”). In some embodiments, the compositions further comprise at least a trace amount of a non-reactive porogen, wherein the non-reactive porogen is impregnated in the nanoporous poly(diol) citrate polymer. In certain embodiments, the compositions further comprise at least a trace amount of polyethylene glycol dimethyl ether (PEGDM), wherein the PEGDM is impregnated in the nanoporous poly(diol) citrate polymer. In other embodiments, the plurality of biologically active molecules are selected from the group consisting of: proteins, nucleic acids, drugs, pro-drugs, and small molecules.

In some embodiments, the present invention provides methods of treating a patient comprising: administering a therapeutic a nanoporous poly(diol) citrate polymer and a plurality of biologically active molecules, where in said nanoporous poly(diol) citrate polymer has the formula (A−B−C−B)ₙ, where A is a linear aliphatic diol, B is citric acid, C is a linear aliphatic diol, and n is an integer greater than 1, and wherein said plurality of biologically active molecules are adsorbed to said nanoporous poly(diol) citrate polymer.

In certain embodiments, the present invention provides novel biocompatible elastomeric polymers that may be used, for example, in tissue engineering. The present invention, in some embodiments, provides methods and compositions for making and using citric acid copolymers. In certain embodiments, there is provided a composition comprising a citric acid polyester having the generic formula (A−B−C)n, wherein A is a linear aliphatic dihydroxy monomer; B is citric acid, C is a linear aliphatic dihydroxy monomer, and n is an integer greater than 1. In specific embodiments, A is a linear diol comprising between about 2 and about 20 carbons. In other embodiments, C is independently a linear diol comprising between about 2 and about 20 carbons. While in certain embodiments, both A and C may be the same linear diol, other embodiments contemplate that A and C are different linear diols. A particularly preferred linear diol is 1,8, octanediol. In other embodiments, one or both ofA and C may be 1,10decanediol. The diol also maybe an unsaturated diol, e.g., tetradeca-2,12-diene-I,14-diol, or other diols including macromonomer diols such as polyethylene oxide, and Nmethyldiethanoamine (MDEA). This family of elastomers is named as poly(diol citrate). In particularly preferred embodiments, the composition of the invention is dihydroxy poly 1,8-octanediol co-citric acid. Poly(diol citrate) can also form hybrids with other materials like hydroxyapatite to form elastomeric composites.

Another aspect of the invention contemplates a substrate that may be formulated for tissue culture and/or tissue engineering wherein the substrate is made of a citric acid polymer as described herein. In preferred embodiments, the substrate may further comprise a surface modification that allows cellular attachment. Preferably, the polymer of the invention employed as cell/tissue culture substrate is biodegradable. Preferably, the polymer also is biocompatible. The “biocompatible” is intended to encompass a polymer that may be implanted in vivo or alternatively may be used for the growth of cells that may be implanted in vivo without producing an adverse reaction, such as an immunological response or otherwise altering the morphology of the cells grown thereon to render the cells incompatible with being implanted in vivo or used to model an in vivo organ.

Also contemplated herein is a method of producing engineered tissue, comprising providing a biodegradable citric acid polymer of the present invention as a scaffold for the growth of cells and culturing cells of said tissue on the scaffold. In preferred methods, the polymer is poly 1,8-octanediol-co-citric acid, or a derivative thereof. In specific embodiments, the cells are selected from the group consisting of endothelial cells, ligament tissue, muscle cells, bone cells, cartilage cells. In other preferred embodiments, the tissue engineering method comprises growing the cells on the scaffold in a bioreactor.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE FIGURES

FIG. 1 is a schematic representation of the synthesis of poly (1,8-octanediol-co-citric acid)

FIG. 2 is an FTIR spectrum of POC

FIG. 3 is a graph depicting stress-strain curves of POC under different reaction conditions

FIG. 4 is a comparison of the stress-strain curves of POC, PDC, PDDC,

PDDCPEO400, POCM and POC-HA.

FIG. 5 is a graph depicting DSC thermograms of POC

FIG. 6 is a graph depicting the contact angle to water vs. time curve of POC.

FIG. 7 is a graph depicting the degradation of POC synthesized under different conditions after incubated in PBS at 37° C. for 6 weeks.

FIG. 8 is a graph depicting weight loss in alkali solution (0.1 M sodium hydroxide aqueous) of POC with or without 5% (monomer mole ratio) glycerol.

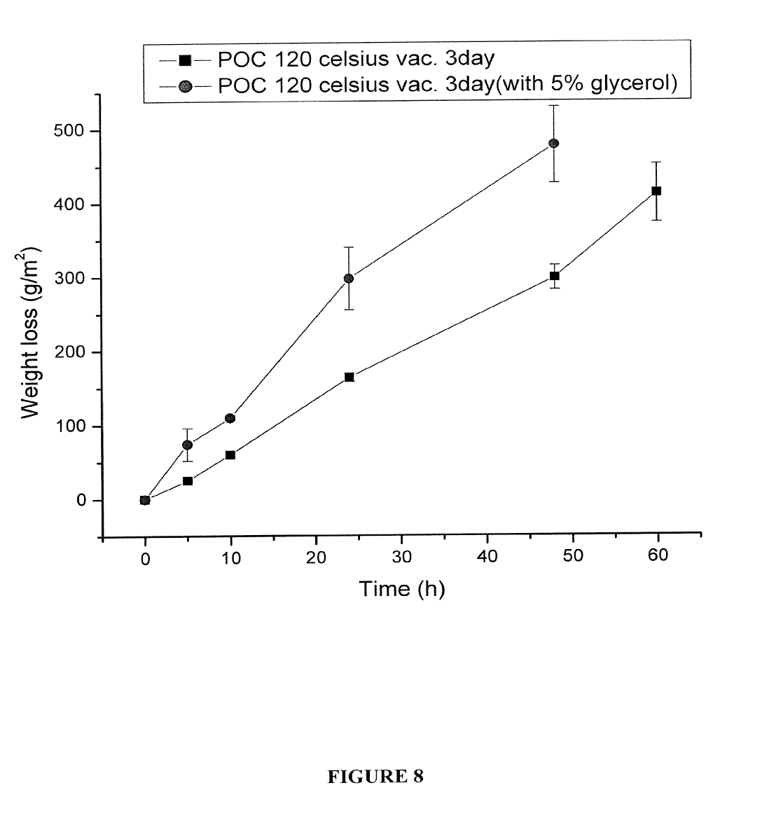

FIG. 9 is a photomicrograph (×100) of human aortic smooth muscle cells on POC at different culture times: A) 1 hour B) 5 hours C) 24 hours and D) 8 days.

FIG. 10 is a graph depicting the results of an MTT-tetrazonium assay of human aortic smooth muscle cells on POC, PLLA (Mw=300,000), and tissue culture polystyrene (TCPS). Formosan absorbance is expressed as a function of culture time.

FIG. 11 is a photomicrograph (×100) (A, B, C, and D) and SEM pictures (E and F) of human aortic endothelial cells on POC at different culture times: A) 1 hour; B) 24 hours: C) 4 days; D) 6 days; E) and F) 6 days.

FIG. 12 is a photomicrograph (×100) of human aortic smooth muscle cells (A) and human aortic endothelial cells (B) on PDC.

FIG. 13 is a photograph depicting porous and non-porous tube scaffold and sponge scaffold made by POC.

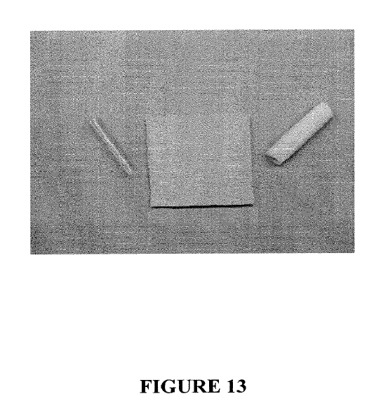

FIGS. 14A and 14B shows graphs depicting the results of wet mechanical tests for POC and PDDC under different conditions. FIG. 14A shows tensile strength and FIG. 14B shows elongation.

FIG. 15 is a schematic drawing depicting a biphasic scaffold.

FIG. 16 shows SEM pictures of A) a cross section of a POC biphasic scaffold; B) the pore structure of the porous phase; C) human aortic smooth muscles cells on the porous phase of co-cultured biphasic scaffold; D) human aortic endothelial cells on the lumen of co-cultured biphasic scaffold.

FIG. 17 The effect of the POC porogen in a 75/25 PEGDM/POC scaffold (500 Mₙ) and a 90/10 NaCl/POC scaffold (100-150 μm NaCl) on pore size distributions as indicated by the percent of total intrusion volume as a function of pore diameter (μm) measured using mercury intrusion porosimetry.

FIG. 18. Representative stress-strain curves for A: control non-porous POC film and B: 75/25 PEGDM/POC nanoporous scaffold.

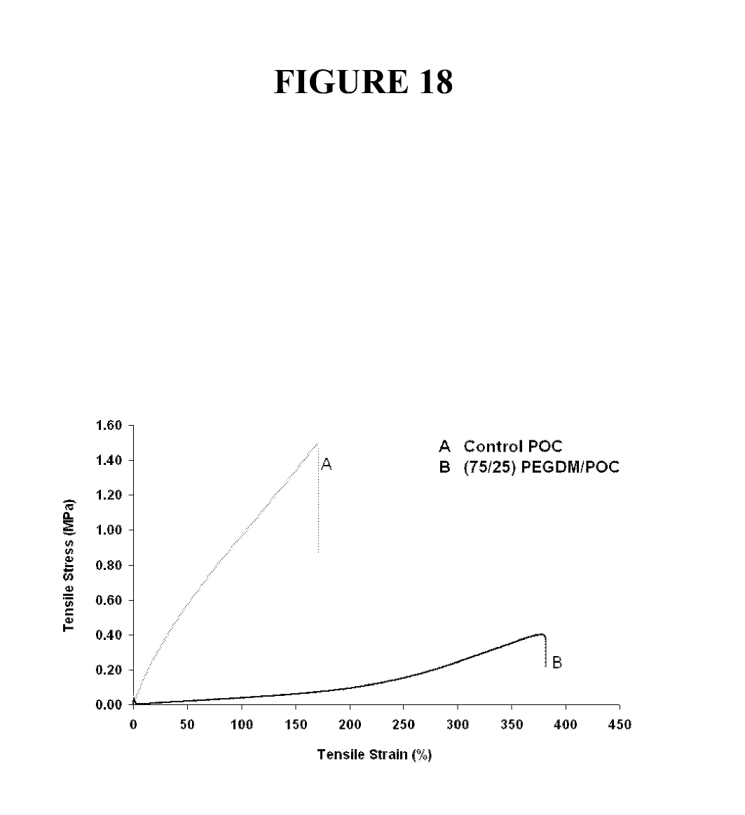

FIG. 19 Degradation studies of “nanoporous POC” and non-porous POC films in (a) PBS (n=6) and (b) 25 mM NaOH solution (n=5) at 37° C. Samples in (a) and (b) include A: nanoporous POC 75/25 PEGDM/POC and B: Control POC film. (Error bars represent SD).

FIG. 20. SEM images of a) Control non leached 50% wt. PEGDM to cross-linked POC polymer and b) same sample that was leached in acetone and critically point dried.

FIG. 21. Polymer FTIR analysis. A) POC (120 C, 2 Pa, t=24 h); B) Pre-polymer POC (t=0 h); C) PEGDM (120 C, 2 Pa, 6=24 h); D) PEGDM (t=0 h); E) 70% by wt. PEGDM in POC (120 C, 2 Pa, t=24 h); F) 70% by wt. PEGDM in POC (t=0 h).

FIG. 22. Drawing depicting the structure of the POC repeating unit and the PEGDM repeating unit.

FIG. 23 shows representative FTIR spectra for pre-polymer POC, PEGDM and sample mixtures of POC and PEGDM before and after polymerization.

FIG. 24. PEGDM leaching in acetone. POC samples which were polymerized containing percent by wt. content PEGDM were leached for 48 hours in multiple changes of acetone and evaluated for mass loss (n=4, error bars represent SD).

FIG. 25. SEM micrographs. A) A non-porous POC control film; B) air-dried 60% by wt. PEGDM showing collapsed POC polymer pores; C) Critically point dried 60% by wt. PEGDM showing intact POC pore structures; D) Critically point dried 70% by wt. PEGDM showing intact POC pore structures on different size scales as the 60% PEGDM sample. (scale bars=4 μm, 8K magnification).

FIG. 26. SEM micrographs. The above images are representative of the polymer pore structure for a 70% by wt. PEGDM sample at different magnifications. A) 200K magnification (scale bar=200 nm) B). 45K magnification (scale bar=1 μm).

FIG. 27. Biotin-dextran in vitro drug release. 80% by wt. PEGDM and control POC films were loaded with biotin-dextran and released at 37° C. in 1×PBS. The cumulative release of biotin-dextran was quantified using a Vitamin H ELISA assay for time points 8 and 24 hours. (n=3, error bars represent SD).

FIG. 28 Mechanical properties. 1) Representative stress-strain curves for A) control POC film containing 0% PEGDM and B) 75% by wt. PEGDM polymer sample. 2) 75% by wt. PEGDM polymer sample before and after breakage from tensile testing.

FIG. 29. In vitro polymer degradation in 1×PBS at 37° C. POC with different percent by wt. PEGDM during polymerization were degraded over the course of two weeks to evaluate the relative degradations rates by modifying the percent content PEGDM. (n=3, SD).

### DESCRIPTION OF THE PREFERRED EMBODIMENTS

The present invention provides citric acid polymers (e.g., elastomeric citric acid polyester polymers) and methods of making and using these citric acid polymers (e.g., as a biologically active molecule delivery platform). In certain embodiments, the citric acid polymer has adsorbed biologically active molecules. In particular embodiments, the citric acid polymer comprises pores that are between about 7 and 15 nanometers in diameter. In other embodiments, the citric acid polymer comprises poly(1,8 octanediol-co-ctric acid). In certain embodiments, the citric acid polymers are made by employing polyethylene glycol dimethyl ether (PEGDM). In certain embodiments, the citric acid polymers are elastomeric polymer (e.g., they have elastic properties).

A. Nanoporous Citrate Polymers

In particular embodiments, the present invention provides the creation of nanoscale pores (<10 nm pore diameter) within a crosslinked polymer network using biodegradable poly(1,8 octanediol-co-citric acid) (POC) polymer in conjunction with a nonreactive porogen, polyethylene glycol dimethyl ether (PEGDM) (500 Mₙ). In certain embodiments, the “nano-porous POC” is synthesized by reacting citric acid and 1,8 octanediol to form covalent cross-linked networks via a polycondensation reaction without exogenous catalysts in the presence of the nonreactive and thermostable porogen, PEGDM. Since cross-linked POC polymer is insoluble in water and organic solvents (i.e. acetone, dioxane, ethanol) and the PEGDM polymer is readily soluble in water and said organic solvents, the PEGDM porogen can be easily removed from the nanoporous POC system through selective leaching in water or mixtures of other organic solvents. Due to the unique properties of nanoscale systems, such as a relatively large surface area to volume ratio, the nanoporous POC can be used as a material to encapsulate and deliver bioactive molecules, protein based drug therapies such as enzymes, and growth factors and other therapeutic agents under very mild and physiologically relevant conditions.

In certain embodiments, the nanoporous poly(diol) citrate polymers of the present invention exploit the use of high porosity with nano-scale pores to adsorb a significant quantity of drugs and/or growth factors under mild conditions (e.g., ≦37° C. in phosphate buffered saline solution) without the use of harsh solvents and fabrication methods which have been shown to denature drug/protein activity. In certain embodiments, the present invention provides fully biodegradable nanoporous poly(diol) citrate elastomer (e.g., nanoporous POC citrate elastomer) for use in tissue engineering and controlled drug delivery applications.

Discussed below are exemplary methods for generating poly(diol) citrate molecules. These methods can be modified to include a non-reactive porogen, such as PEGDM, to generate nanoporous poly(diol) citrate molecules. Example 1 below provides teachings of how to introduce nanopores into biocompatible membranes made from such poly(diol) citrate molecules. These nanoporous materials described herein can be used in, for example, tissue engineering, drug delivery, or any application where porous biodegradable and flexible elastomeric materials may be necessary. The increasing and fast development of tissue engineering applications will require this type of technology to maximize flexibility for design requirements of scaffolds for tissue engineering or drug delivery devices.

Guidance on generating poly(diol) citrate polymers is found in the art, for example, in U.S. Patent Publications 20070071790 and 20050063939, which are both herein incorporated by reference in their entireties as if fully set forth herein. Exemplary guidance on making such poly(diol) citrate polymers is also provided briefly below. Suck poly(diol) citrate polymers can be made with nonreactive porogens (as described in Example 1) to generate nanoporous poly(diol) citrate polymers.

Biodegradable elastomeric polymers of poly(diol) citrate molecules are described in Pat. Pubs. 20070071790 and 20050063939. Such molecules typically comprise a polyester network of citric acid copolymerized with a linear aliphatic di-OH monomer in which the number of carbon atoms ranges from 2 to 20 (e.g., 2, 3, 4, 5, 6, 7, 8, 9, 10, 11, 12, 13, 14, 15, 16, 17, 18, 19, or 20). Polymer synthesis conditions for the preparation of these molecules vary from mild conditions, even at low temperature (less than 100° C.) and no vacuum, to tough conditions (high temperature and high vacuum) according the requirements for the materials properties. By changing the synthesis conditions (including, but not limited to, post-polymerization temperature, time, vacuum, the initial monomer molar ratio, and the di-OH monomer chain length) the mechanical properties of the polymer can be modulated over a wide range. This series of polymers exhibit a soft, tough, biodegradable, hydrophilic properties and excellent biocompatibility in vitro.

Poly(diol)citrate polymers can, for example, be described by the following general structure: (A−B−C−B)ₙ, where A is a linear, aliphatic diol and C also is a linear aliphatic diol, B is citric acid. The citric acid co-polymers can be made up of multiples of the above formula, as defined by the integer n, which may be any integer greater than 1. In certain embodiments, n may range from 1 to about 1000 or more. For example, n may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 31, 32, 33, 34, 35, 36, 37, 38, 39, 40, 41, 42, 43, 44, 45, 46, 47, 48, 49, 50, or more.

In certain embodiments, the identity of “A” above is poly 1,10-decanediol and in another embodiment the identity of A is 1,8-octanediol. However, it should be understood that this is merely an exemplary linear, aliphatic diol. Those of skill are aware of other aliphatic alcohols that will be useful in polycondensation reactions to produce poly citric acid polymers. Exemplary such aliphatic diols include any diols of between about 2 carbons and about 20 carbons. While the diols are preferably aliphatic, linear, unsaturated diols, with the hydroxyl moiety being present at the C₁ and Cₓ position (where x is the terminal carbon of the diol), it is contemplated that the diol may be an unsaturated diol in which the aliphatic chain contains one or more double bonds. The identity for “C” in one embodiment is 1,8, octanediol, however as with moiety “A,” “C” may be any other aliphatic alcohols. While in specific embodiments, both A and C are both the same diol, e.g., 1,8-octanediol, it should be understood that A and C may have different carbon lengths. For example, A maybe 2, 3, 4, 5, 6, 7, 8, 9, 10, 11, 12, 13, 14, 15, 16, 17, 18, 19, 20 or more carbons in length, and C may independently be 2, 3, 4, 5, 6, 7, 8, 9, 10, 11, 12, 13, 14, 15, 16, 17, 18, 19, 20 ormore carbons in length. Exemplary methods for the polycondensation of the citric acid with the linear diols are provided below.

Synthesis of Poly(1,10-decanediol-co-citric acid) (PDC) In a typical procedure, 19.212 g citric acid and 17.428 g 1,10-decanediol are added to a 250 ml three-neck round-bottom flask, fitted with an inlet adapter and an outlet adapter. The mixture is then melted within 15 min by stirring at 160-165° C. in silicon oil bath, and then the temperature of the system was lowered to 120° C. The mixture is stirred for half an hour at 120° C. to get the pre-polymer. Nitrogen is vented throughout the above procedures. The pre-polymer is post-polymerized at 60° C., 80° C. or 120° C. with and without vacuum for predetermined time from one day to 3 weeks depending on the temperature to achieve the Poly(1,10-decanediol-co-citric acid). Nitrogen is introduced into the reaction system before the polymer was taken out from reaction system.

Preparation of Poly(1,8-Octanediol-co-citric acid) (POC) In a typical procedure, 19.212 g citric acid and 14.623 g Octanediol are added to a 250 mL three-neck round-bottom flask, fitted with an inlet adapter and an outlet adapter. The mixture is melted within 15 min by stirring at 160-165° C. in silicon oil bath, and then the temperature of the system was lowered to 140° C. The mixture is stirred for another 1 hr at 140° C. to get the pre-polymer. Nitrogen is vented throughout the above procedures. The pre-polymer is post-polymerized at 60° C., 80° C. or 120° C. with and without vacuum for predetermined time (from one day to 3 weeks depending on the temperature, with the lower temperatures requiring longer times) to achieve the Poly(1,8-octanediol-co-citric acid). Nitrogen is introduced into the reaction system before the polymer was taken out from reaction system.

Porous scaffolds of POC (tubular and flat sheets) may be prepared via a salt leaching technique as follows: POC pre-polymer is dissolved into dioxane to form 25 wt % solution, and then the sieved salt (90-120 microns) is added into pre-polymer solution to serve as a porogen. The resulting slurry is cast into a poly(tetrafluoroethylene) (PTFE) mold (square and tubular shape). After solvent evaporation for 72 h, the mold is transferred into a vacuum oven for post-polymerization. The salt in the resulting composite is leached out by successive incubations in water (produced by Milli-Q water purification system) every 12 h for a total 96 h. The resulting porous scaffold is air-dried for 24 hr and then vacuum dried for another 24 hrs. The resulting scaffold is stored in a dessicator under vacuum before use. Porous scaffolds are typically preferred when cells are expected to migrate through a 3-dimensional space in order to create a tissue slice. Solid films may be used when a homogenous surface or substrate for cell growth is required such as an endothelial cell monolayer within the lumen of a vascular graft Using similar techniques porous scaffold of PDC or other poly(diol)citrates can be prepared.

Synthesis of Poly(1,6-hexanediol-co-citric acid) (PHC). In a typical procedure, 19.212 g citric acid and 11.817 g 1,6-hexanediol were added to a 250 ml three-neck round-bottom flask, fitted with an inlet adapter and an outlet adapter. The mixture is melted within 15 min by stirring at 160-165° C. in a silicon oil bath, and then the temperature of the system was lowered to 120° C. The mixture is stirred for half an hour at 120° C. to get the pre-polymer. Nitrogen is vented throughout the above procedures. The pre-polymer is post-polymerized at 60° C., 80° C. or 120° C. with and without vacuum for a predetermined time from one day to 3 weeks, depending on the temperature, to achieve the Poly(1,6-hexanediol-co-citric acid). Nitrogen is introduced into the reaction system before the polymer was taken out from reaction system.

Synthesis of Poly(1,12-dodecanediol-co-citric acid) PDDC. In a typical experiment, 19.212 g citric acid and 20.234 g 1,12-dodecanediol were added to a 250 ml three-neck round-bottom flask, fitted with an inlet adapter and an outlet adapter. The mixture is melted within 15 min by stirring at 160-165° C. in silicon oil bath, and then the temperature of the system is lowered to 120° C. The mixture is stirred for half an hour at 120° C. to get the pre-polymer. Nitrogen is vented throughout the above procedures. The pre-polymer is post-polymerized at 60° C., 80° C. or 120° C. with and without vacuum for predetermined time from one day to 3 weeks depending on the temperature to achieve the Poly(1,12-dodecanediol-co-citric acid). Nitrogen is introduced into the reaction system before the polymer is taken out from reaction system.

Synthesis of Poly(1,8-octanediol-co-citric acid-co-glycerol) In a typical procedure (Poly(1,8-octanediol-co-citric acid-co-1% glycerol), 23.0544 g citric acid, 16.5154 g 1,8-octanediol and 0.2167 g glycerol is added to a 250 ml three-neck round-bottom flask, fitted with an inlet adapter and an outlet adapter. The mixture is melted within 15 min by stirring at 160-165° C. in silicon oil bath, and then the temperature of the system is lowered to 120° C. The mixture is stirred for another hour at 140° C. to get the pre-polymer. Nitrogen is vented throughout the above procedures. The pre-polymer is post-polymerized at 60° C., 80° C. or 120° C. with and without vacuum for predetermined time from one day to 3 weeks depending on the temperature to achieve the Poly(1,8-octanediol-co-citric acid-co-1% glycerol). Nitrogen is introduced into the reaction system before the polymer is taken out from reaction system.

Synthesis of Poly(1,8-octanediol-citric acid-co-polyethylene oxide). In a typical procedure, 38.424 g citric acid, 14.623 g 1,8-octanediol and 40 g polyethylene oxide with molecular weight 400 (PEO400)(100 g PEO1000 and 200 g PEO2000 respectively) (molar ratio: citric acid/1,8-octanediol/PEO400=1/0.5/0.5) is added to a 250 ml or 500 ml three-neck round-bottom flask, fitted with an inlet adapter and an outlet adapter. The mixture is melted within 15 min by stirring at 160-165° C. in silicon oil bath, and then the temperature of the system is lowered to 135° C. The mixture is stirred for 2 hours at 135° C. to get the pre-polymer. Nitrogen is vented throughout the above procedures. The pre-polymer is post-polymerized at 120° C. under vacuum for predetermined time from one day to 3 days to achieve the Poly(1,8-octanediol-citric acid-co-polyethylene oxide). Nitrogen is introduced into the reaction system before the polymer is taken out from reaction system. The molar ratios can be altered to achieve a series of polymers with different properties.

Synthesis of Poly(1,12-dodecanediol-citric acid-co-polyethylene oxide). In a typical procedure, 38.424 g citric acid, 20.234 g 1,12-dodecanediol and 40 g polyethylene oxide with molecular weight 400 (PEO400)(100 g PEO1000 and 200 g PEO2000 respectively) (molar ratio: citric acid/1,8-octanediol/PEO400=1/0.5/0.5) is added to a 250 ml or 500 ml three-neck round-bottom flask, fitted with an inlet adapter and an outlet adapter. The mixture is melted within 15 min by stirring at 160-165° C. in silicon oil bath, and then the temperature of the system is lowered to 120° C. The mixture is stirred for half an hour at 120° C. to get the pre-polymer. Nitrogen is vented throughout the above procedures. The pre-polymer is post-polymerized at 120° C. under vacuum for predetermined time from one day to 3 days to achieve the Poly(1,12-dodecanediol-citric acid-co-polyethylene oxide). Nitrogen is introduced into the reaction system before the polymer was taken out from reaction system. The molar ratios can be altered to achieve a series of polymers with different properties.

Synthesis of Poly(1,8-octanediol-citric acid-co-N-methyldiethanoamine) POCM.

In a typical experiment, 38.424 g citric acid, 26.321 g 1,8-octanediol and 2.3832 g N-methyldiethanoamine (MDEA) (molar ratio: citric acid/1,8-octanediol/MDEA=1/0.90/0.10) were added to a 250 ml or 500 ml three-neck round-bottom flask, fitted with an inlet adapter and an outlet adapter. The mixture is melted within 15 min by stirring at 160-165° C. in silicon oil bath, and then the temperature of the system is lowered to 120° C. The mixture is stirred for half an hour at 120° C. to get the pre-polymer. Nitrogen is vented throughout the above procedures. The pre-polymer was post-polymerized at 80° C. for 6 hours, 120° C. for 4 hours without vacuum and then 120° C. for 14 hours under vacuum to achieve the Poly(1,8-octanediol-citric acid-co-N-methyldiethanoamine). Nitrogen is introduced into the reaction system before the polymer is taken out from reaction system. The molar ratios can be altered to citric acid/1,8-octanediol/MDEA=1/0.95/0.05.

Synthesis of Poly(1,12-dodecanediol-citric acid-co-N-methyldiethanoamine) PDDCM. In a typical procedure, 38.424 g citric acid, 36.421 g 1,12-dodecanediol and 2.3832 g N-methyldiethanoamine (MDEA) (molar ratio: citric acid/1,8-octanediol/MDEA=1/0.90/0.10) is added to a 250 ml or 500 ml three-neck round-bottom flask, fitted with an inlet adapter and an outlet adapter. The mixture is melted within 15 min by stirring at 160-165° C. in a silicon oil bath, and then the temperature of the system is lowered to 120° C. The mixture is stirred for half an hour at 120° C. to get the pre-polymer. Nitrogen is vented throughout the above procedures. The pre-polymer is post-polymerized at 80° C. for 6 hours, 120° C. for 4 hours without vacuum and then 120° C. for 14 hours under vacuum to achieve the Poly(1,12-dodecanediol-citric acid-co-N-methyldiethanoamine). Nitrogen is introduced into the reaction system before the polymer is taken out from reaction system. The molar ratios can be altered to citric acid/1,12-dodecanediol/MDEA=1/0.95/0.05.

B. New Biodegradable Elastomeric Polymers

In certain embodiments, described in the present specification are a family of novel biodegradable elastomeric polymers comprising a polyester network of citric acid copolymerized with a linear aliphatic di-OH monomer in which the number of carbon atoms ranges from 2 to 20. Polymer synthesis conditions vary from mild conditions, even at low temperature (less than 100° C.) and no vacuum, to tough conditions (high temperature and high vacuum) according the requirements for the materials properties. By changing the synthesis conditions (including, but not limited to, post polymerization temperature, time, vacuum, the initial monomer molar ratio, and the di-OH monomer chain length) the mechanical properties of the polymer can be modulated over a wide range. This series of polymers exhibit a soft, tough, biodegradable, hydrophilic properties and excellent biocompatibility in vitro.

In certain embodiments, the polymers of the present invention have a general structure of:

(A−B−C)n

where A is a linear, aliphatic diol and C also is a linear aliphatic diol. B is citric acid. In particular embodiments, the citric acid co-polymers of the present invention are made up of multiples of the above formula, as defined by the integer n, which may be any integer greater than 1. It is contemplated that n may range from 1 to about 1000 or more. It is particularly contemplated that n may be 1,2,3,4,5,6, 7, 8, 9, 10, 11, 12, 13, 14, 15,16,17, 18, 19,20,21,22,23,24,25,26,27,28,29,3˜31,31,32,33,34,35,36, 37, 38, 39, 40,41,42,43, 44, 45, 46,47,48,49, 50, or more.

In preferred embodiments, the identity of “A” above is 1,8-octanediol. However, it should be understood that this is merely an exemplary linear, aliphatic diol. Those of skill are aware of other aliphatic alcohols that will be useful in polycondensation reactions to produce poly citric acid polymers. Exemplary such aliphatic diols include any diols of between about 2 carbons and about 20 carbons. While the diols are preferably aliphatic, linear, unsaturated diols, with the hydroxyl moiety being present at the C1 and Cx position (where x is the terminal carbon of the diol), it is contemplated that the diol may be an unsaturated diol in which the aliphatic chain contains one or more double bonds. The preferred identity for “c” in one embodiment is 1,8, octanediol, however as with moiety “A,” “c” may be any other aliphatic alcohols. While in specific embodiments, both A and C are both the same diol, e.g., 1,8-octanediol, it should be understood that A and C may have different carbon lengths. For example, A may be 2,3,4,5,6, 7, 8, 9, 10, 11, 12, 13, 14, 15, 16, 17, 18, 19, 20 ormore carbons in length, and C may independently be 2,3,4,5,6, 7, 8,9, 10, 11, 12, 13, 14, 15, 16, 17, 18, 19,20 or more carbons in length. Exemplary methods for the polycondensation of the citric acid with the linear diols are provided herein below in the Examples.

The polymers of present invention may be utilized to form hybrids with other materials to form elastomeric composites. In those embodiments where the other materials are used, the other materials can be in-organic materials, polymers with any kind of forms such as powder, fiber, and films. The other materials can also be elastomeric or non-elastomeric. In a particularly embodiment, the elastomeric composite can be a hybrid of the polymers of present invention with hydroxyapatite (POC-HA).

The polymers of the present invention may be useful both as substrata for the growth and propagation of tissues cells that may be seeded on the substrata and also as implantable devices. In those embodiments where the polymers are used as bioimplantable devices, the substrate may be formulated into a shape suitable for implantation. For example, as described in U.S. Pat. No. 6,620,203 (incorporated herein by reference), it may be desirable to produce prosthetic organ tissue for implantation into an animal, such as e.g., testicular tissue described in the U.S. Pat. No. 6,620,203 patent. Other organs for which tissue implantation patches may be generated include, but are not limited to skin tissue for skin grafts, myocardial tissue, bone tissue for bone regeneration, testicular tissue, endothelial cells, blood vessels, and any other cells from which a tissue patch may be generated. Thus, those of skill in the art would understand that the aforementioned organs/cells are merely exemplary organs/cell types and it should be understood that cells from any organ may be seeded onto the biocompatible polymers of the invention to produce useful tissue for implantation and/or study.

The cells that may be seeded onto the polymers of the present invention may be derived from commercially available cell lines, or alternatively may be primary cells, which can be isolated from a given tissue by disaggregating an appropriate organ or tissue which is to serve as the source of the cells being grown. This may be readily accomplished using techniques known to those skilled in the art. Such techniques include disaggregation through the use of mechanically forces either alone or in combination with digestive enzymes and/or chelating agents that weaken cell-cell connections between neighboring cells to make it possible to disperse the tissue into a suspension of individual cells without appreciable cell breakage. Enzymatic dissociation can be accomplished by mincing the tissue and treating the minced tissue with any of a number of digestive enzymes either alone or in combination. Digestive enzymes include but are not limited to trypsin, chymotrypsin, collagenase, elastase, and/or hyaluronidase, Dnase, pronase, etc. Mechanical disruption can also be accomplished by a number of methods including, but not limited to the use of grinders, blenders, sieves, homogenizers, pressure cells, or sonicators to name but a few. For a review of tissue disaggregation techniques, see Freshney, Culture of Animal Cells. A Manual of Basic Technique, 2d Ed., A. R. Liss, Inc., New York, 1987, Ch. 9, pp. 107-126.

Once the primary cells are disaggregated, the cells are separated into individual cell types using techniques known to those of skill in the art. For a review of clonal selection and cell separation techniques, see Freshney, Culture of Animal Cells. A Manual of basic Techniques, 2d Ed., A. R. Liss, Inc., New York, 1987, Ch. 11 and 12, pp. 137-168. Media and buffer conditions for growth of the cells will depend on the type of cell and such conditions are known to those of skill in the art. In certain embodiments, it is contemplated that the cells attached to the biocompatible polymeric substrates of the invention are grown in bioreactors. A bioreactor may be of any class, size or have anyone or number of desired features, depending on the product to be achieved. Different types of bioreactors include tank bioreactors, immobilized cell bioreactors, hollow fiber and membrane bioreactors as well as digesters. There are three classes of immobilized bioreactors, which allow cells to be grown: membrane bioreactors, filter or mesh bioreactors, and carrier particle systems. Membrane bioreactors grow the cells on or behind a permeable membrane, allowing the nutrients to leave the cell, while preventing the cells from escaping. Filter or mesh bioreactors grow the cells on an open mesh of an inert material, allowing the culture medium to flow past, while preventing the cells from escaping. Carrier particle systems grow the cells on something very small, such as small nylon or gelatin beads. The bioreactor can be a fluidized bed or a solid bed. Other types of bioreactors include pond reactors and tower fermentors. Any of these bioreactors may be used in the present application for regenerating/engineering tissues on the citric acid polymers of the present invention.

Certain tissues that are regenerated by use of the citric acid polymers of the invention may be encapsulated so as to allow the release of release of desired biological materials produced by the cells at the site of implantation, while sequestering the implanted cells from the surrounding site. Cell encapsulation can be applied to all cell types secreting a bioactive substance either naturally or through genetic engineering means. In practice, the main work has been performed with insulin secreting tissue.

Encapsulation procedures are most commonly distinguished by their geometrical appearance, i.e. micro- or macro-capsules. Typically, in microencapsulation, the cells are sequestered in a small permselective spherical container, whereas in macroencapsulation the cells are entrapped in a larger nonspherical membrane, Lim et al. (U.S. Pat. Nos. 4,409,331 and 4,352,883) discloses the use of microencapsulation methods to produce biological materials generated by cells in vitro, wherein the capsules have varying permeabilities depending upon the biological materials of interest being produced, Wu et aI, Int. J. Pancreatology, 3:91100 (1988), disclose the transplantation of insulin-producing, microencapsulated pancreatic islets into diabetic rats.

As indicated above, the cells that are seeded on the polymers of the present invention may be cell lines or primary cells. In certain preferred embodiments, the cells are genetically engineered cells that have been modified to express a biologically active or therapeutically effective protein product. Techniques for modifying cells to produce the recombinant expression of such protein products are well known to those of skill in the art.

### Example 1

### Preparation of Poly(1,8-Octanediol-Co-Citric acid) (POC)

In a typical experiment, 19.212 g citric acid and 14.623 g Octanediol were added to a 250 mL three-neck round-bottom flask, fitted with an inlet adapter and an outlet adapter. The mixture was melted within 15 min by stirring at 160-165° C. in silicon oil bath, and then the temperature of the system was lowered to 140° C. The mixture was stirred for another 1 hr at 140° C. to get the pre-polymer. Nitrogen was vented throughout the above procedures. The pre-polymer was post- polymerized at 60° C., 80° C. or 120° C. with and without vacuum for predetermined time (from one day to 3 weeks depending on the temperature, with the lower temperatures requiring longer times) to achieve the Poly (1,8-octanediol-co-citric acid). Nitrogen was introduced into the reaction system before the polymer was taken out from reaction system.

Porous scaffolds of POC (tubular and flat sheets) were prepared via a salt leaching technique. Briefly, sodium chloride salt was ground up and sieved for particle sizes between 90 and 125 microns. The salt particles are then mixed with the pre-polymer solution to the desired mass fraction to obtain a corresponding porosity. Typically, the mass fraction of the salt particles will result in a similar % porosity.

### Example 2

### Preparation of Porous Scaffolds of POC

Porous scaffolds of POC (tubular and flat sheets) were prepared via a salt leaching technique as follows: POC pre-polymer was dissolved into dioxane to form 25 wt % solution, and then the sieved salt (90-120 microns) was added into pre-polymer solution to serve as a porogen. The resulting slurry was cast into a poly(tetrafluoroethylene) (PTFE) mold (square and tubular shape). After solvent evaporation for 72 h, the mold was transferred into a vacuum oven for post-polymerization. The salt in the resulting composite was leached out by successive incubations in water (produced by Milli-Q water purification system every 12 h for a total 96 h. The resulting porous scaffold was air-dried for 24 hr and then vacuum dried for another 24 hrs. The resulting scaffold was stored in a dessicator under vacuum before use. Porous scaffolds are typically preferred when cells are expected to migrate through a 3-dimensional space in order to create a tissue slice. Solid films would be used when a homogenous surface or substrate for cell growth is required such as an endothelial cell monolayer within the lumen of a vascular graft.

### Example 3

### Characterization of POC

The following Example provides details of methods and results of characterization of POC.

### Methods

Fourier transform infrared (FTIR) spectroscopy measurements. Infrared spectra were recorded on a Biorad FTS40 Fourier transform infrared spectrometer. Sample POC films with thickness of 12-16 microns were prepared from POC solid samples using a Microtome.

Mechanical Tests. Tensile tests were conducted according to ASTM D412a on an Instron 5544 mechanical tester equipped with 500 N load cell. The POC sample size was 26×4×1.5 mm.

Differential scanning calorimetry (DSC) measurements. Differential scanning calorimetry thermograms were recorded in the range of −80 to 600° C. on a DSC550 (Instrument Specialists Inc.) instrument at a heating rate of 10° C./min.

In vitro degradation. The disk specimen (7 mm in diameter, about 1 to 1.5 mm thickness) was placed in a small container containing 10 ml phosphate buffer saline (pH 7.4). The container was incubated at 37° C. for various times. After incubation the disk was washed with water and dried under vacuum for one week. The mass loss was calculated by comparing the initial mass (W0) with that a given time point (WJ), as shown in Eq. (1). Three individual experiments were performed in triplicate for the degradation test. The results are presented as means±standard deviation (n=3).

Mass loss (%)=[(W0−W.)/W0]×100 (1)

Alkali hydrolysis. Alkali hydrolysis of the disk specimen (8.5 mm in diameter, about 1 to 1.5 mm thickness) was conducted in a 0.1 M sodium hydroxide aqueous solution at 37° C. for various times. The degree of degradation was estimated from the weight loss expressed as g/m2, which was calculated by dividing the weight loss by the total surface area of the disk.

Cell culture. Human aortic smooth muscle and endothelial cells (Clonetics) were cultured in a 50 ml culture flask with SmGM-2 and EBM-2 culture medium (Clonetics). Cell culture was maintained in a water-jacket incubator equilibrated with 5% CO2 at 37° C. When the cells had grown to confluence, the cells were passaged using a Subculture Reagent Kit (Clonetics). Polymer films were cut into small pieces (1.times.2 cm1 and placed in cell culture dishes (6 cm in diameter). Polymer films were sterilized in 70% ethanol and the ethanol was exchanged with an excess amount of phosphate-buffered saline (PBS). The PBS was removed with a pipette and then the samples were sterilized UV light for another 30 min. A 5 ml cell suspension with 6.6×10⁴/ml was added to the culture dish. The morphology of cell attachment was observed and photographed with an inverted light microscope (Nikon Eclipse, TE2000-U) equipped with a Photometrics CooISNAP HQ after culturing a predetermined time. After reaching confluence, the samples were fixed by 2.5% glutaraldehyde solution and dehydrated sequentially in 50, 70, 95 and 100% ethanol each for 10 min. The fixed samples were lyophilized, sputter-coated with gold and examined under scanning electron microscope (SEM, Hitachi 3500N). Polymer films were cut into small disks (7 mm in diameter) with the aid of a cork borer in order to locate the disks into a 96-well tissue culture plate. PLLA films and Tissue culture polystyrene (TCPS) were used as control. The samples were sterilized as described above. The human aortic smooth muscle cells (3.13×10³/well) were added to the wells. The viability and proliferation of the cells were determined by MTT assays. The absorbance of produced Formosan was measured at 570 nm using microplate reader (Tecan, SAFFIRE).

### Results

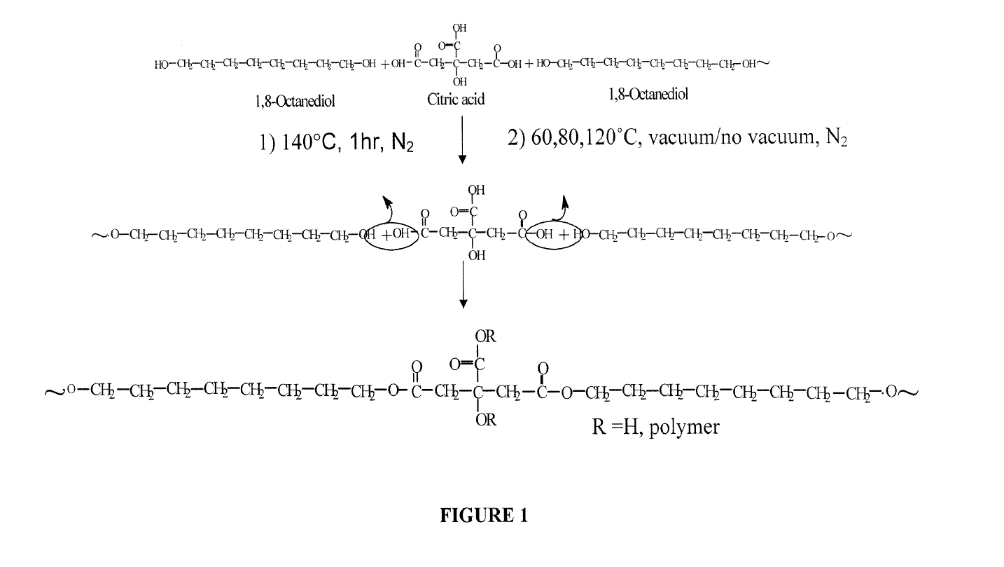

Polycondensation of citric acid and 1,8-octanediol yields a transparent film. The resulting polymer features a small number of crosslinks and carboxyl and hydroxyl groups directly attached to the polymer backbone (FIG. 1).

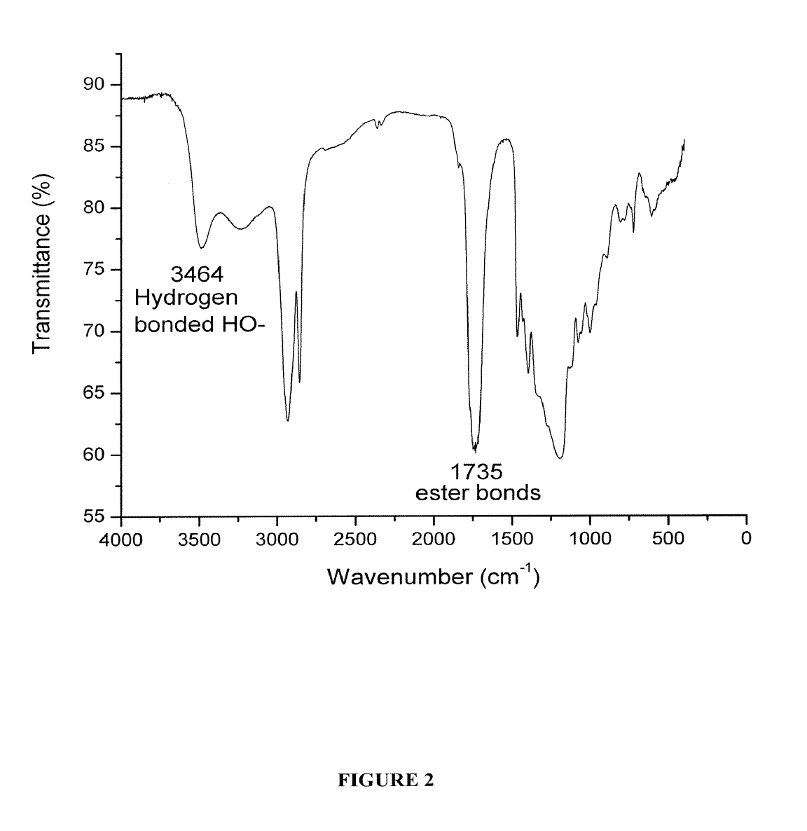

The typical FTIR spectrum of a POC preparation is shown in FIG. 2. The intense C═O stretch at 1,735 cm-1 in FTIR spectrum confirms the formation of ester bonds. The intense OH stretch at 3,464 cm-1 indicates that the hydroxyl groups are hydrogen bonded.

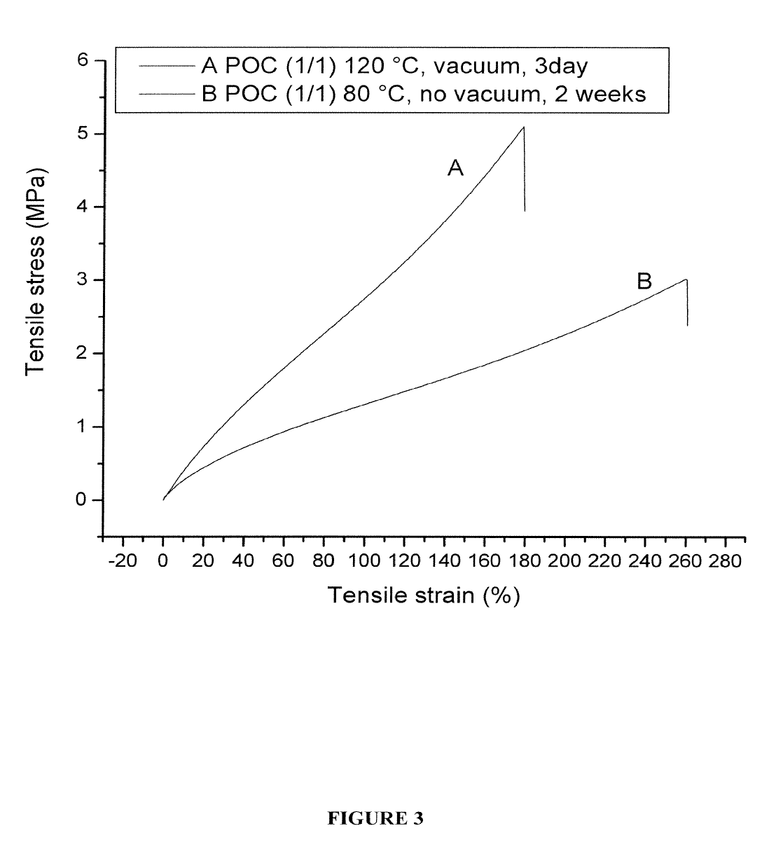

Tensile tests on strips of POC prepared under different synthetic conditions reveal a stress-strain curve characteristic of an elastomeric and tough material (FIG. 3). The nonlinear shape of the tensile stress-strain curve, low modulus and large elongation ratio is typical for elastomers and resembles those of ligament and vulcanized rubber [4]. These results further demonstrate that when the post-polymerization reaction is carried out under lower temperature, the resulting polymer is more elastic than when it is performed at higher temperatures. Post polymerization at lower temperature under vacuum (i.e. 40° C.) may enable incorporation of biological molecules within POC without significant loss of biological activity. Tissue engineering applications that require significant elasticity and strength such as for vascular grafts and heart valves may benefit from post-polymerization at the lower conditions. Tissue engineering applications that require a more rigid or stiff scaffold such as cartilage tissue engineering would benefit from post-polymerization at the higher temperatures.

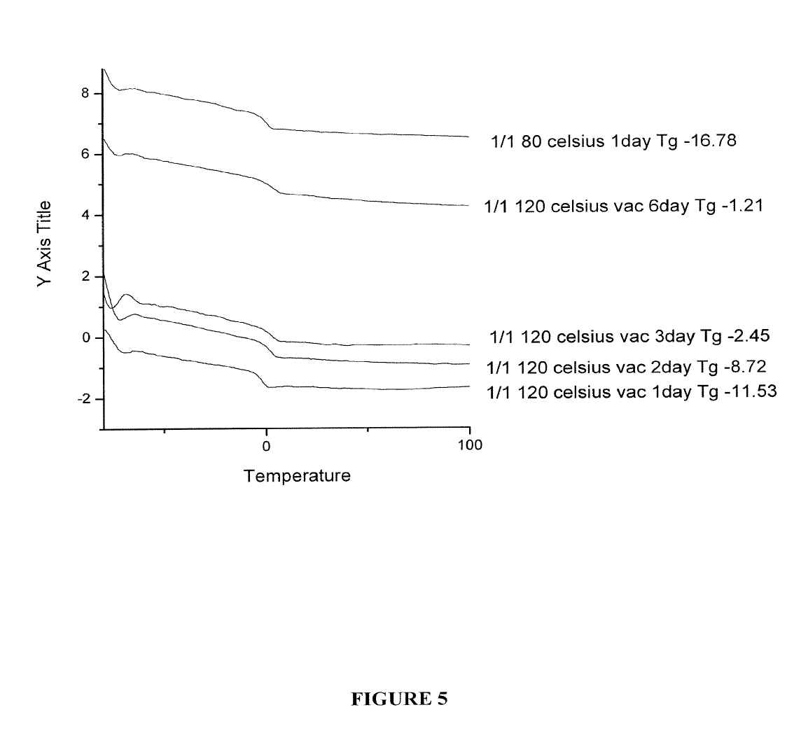

The thermal properties of POC were investigated by DSC. From the thermograms depicted in FIG. 5, no crystallization temperature and melting temperature are observed and apparent glass transition temperature (Tg) is observed below 0° C. for POC synthesized under a variety of conditions. This result shows POC is totally amorphous at 37° C. similar to the vulcanized rubber. FIG. 5 shows the Tg changes with the synthesis conditions. Increasing post-polymerization temperature and elongating the treating time can increase the crosslinking density and then result in the increase of Tg. The Tg is still significantly below 37° C., making the material elastomeric for tissue engineering applications that require elastomeric scaffolds (i.e. cardiovascular, pulmonary, ligament tissue engineering). This result also confirms that POC is a cross-linked polymer. Similar results were observed with PDC.

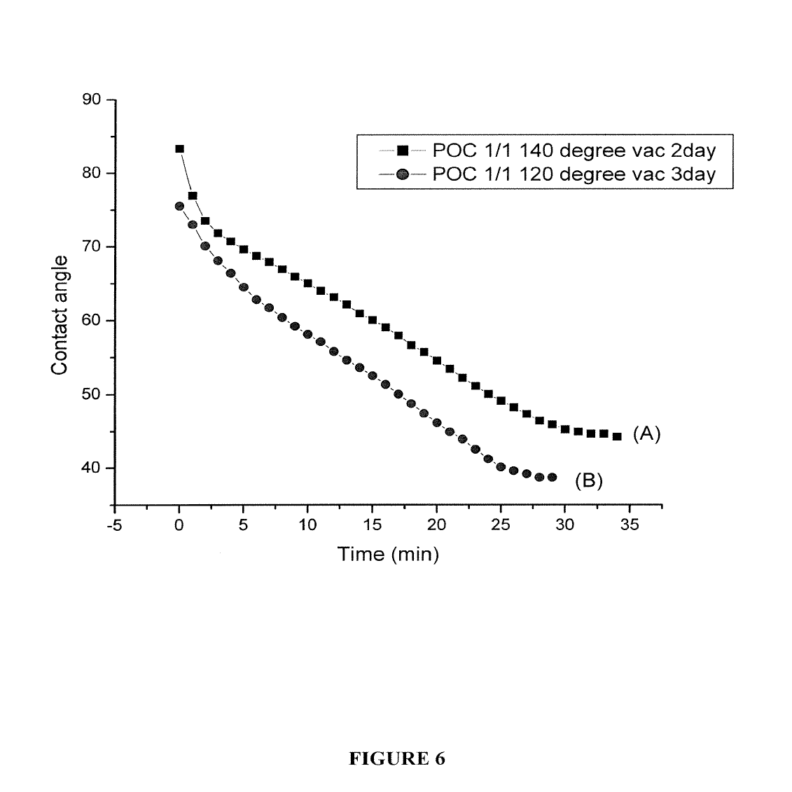

FIG. 6 shows the contact angle to water vs. time curve of POC. The initial contact angle of the POC synthesized under different conditions is 76° and 84°, respectively. The water drop spread out with the time. The contact angles finally reach 38° and 44°, respectively. Although the initial contact angle is relatively high, the polymer chains are highly mobile since POC is a rubber-like and amorphous polymer at room temperature, and the polar water molecules can induce the polar groups such as hydroxyl and carboxyl to enrich at the polymer surface via surface rearrangement. The results show POC is a hydrophilic polymer. Hydrophilic polymers are expected to promote endothelial cell adhesion and proliferation as presented in preliminary data.

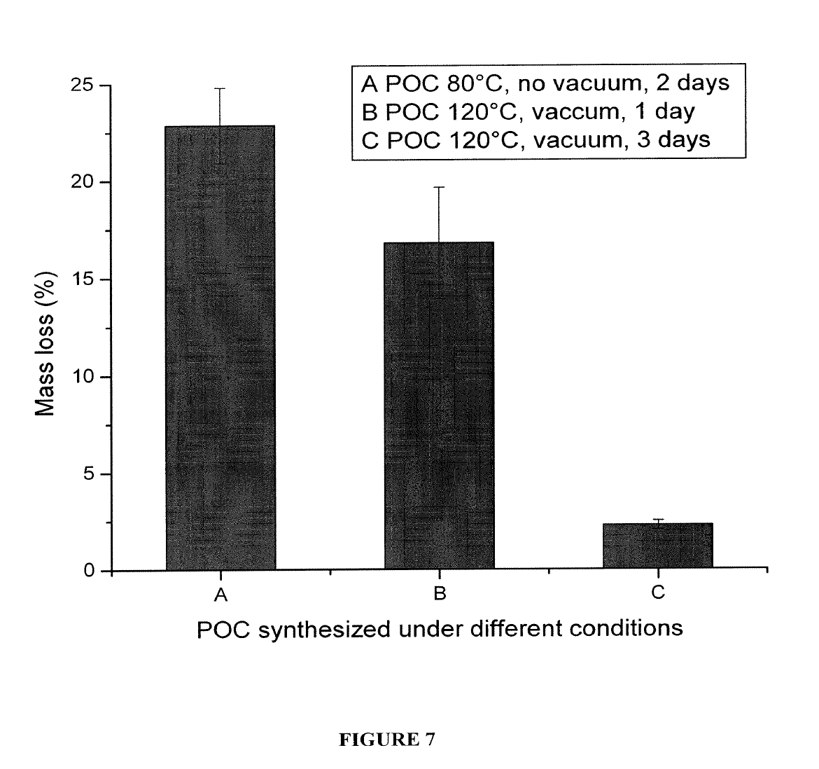

FIG. 7 shows the degradation of POC synthesized under different conditions after incubation in PBS at 37° C. for 6 weeks. POC synthesized under mild conditions (A) has a faster degradation rate compared to that of POC synthesized under relatively tougher conditions (B and C). The degradation rate of POC (B) is considerably faster than that of POC (C). POC synthesized under tough conditions features a high cross-linking degree and the penetration of water molecules into the network films is difficult because of the smaller network space. This is the reason why the degradation rate sequence is POC (A)>POC (B)>POC (C). These results show that POC is degradable polymer. The degradation rate can be modulated by changing synthesis conditions.

In order to achieve better control for the degradation of “highly cross-linked” POC, a third monomer, glycerol is added in addition to the citric acid and diol monomer (0-3 mol %, the molar ratio of carboxyl and hydroxyl group among the three monomers was maintained as 1/1). Increasing amounts of glycerol will result in an increased break strength and Young's modulus. The alkali hydrolysis results show that the addition of glycerol can enhance the degradation of POC in alkali solution. Glycerol is a hydrophilic component. Its addition can facilitate the water penetration into the network films which results in the faster degradation rate.

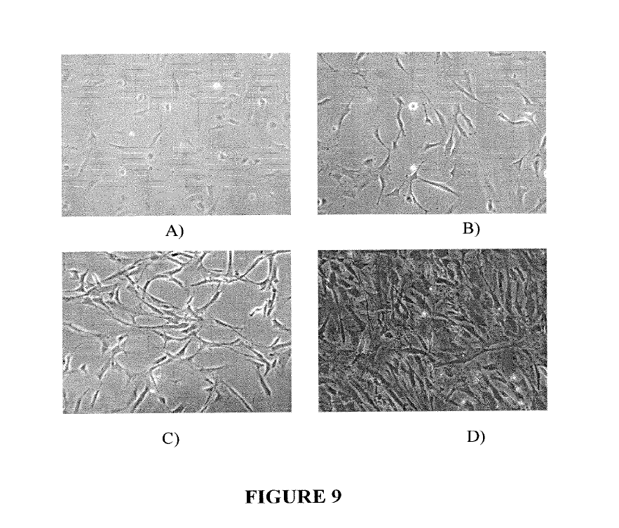

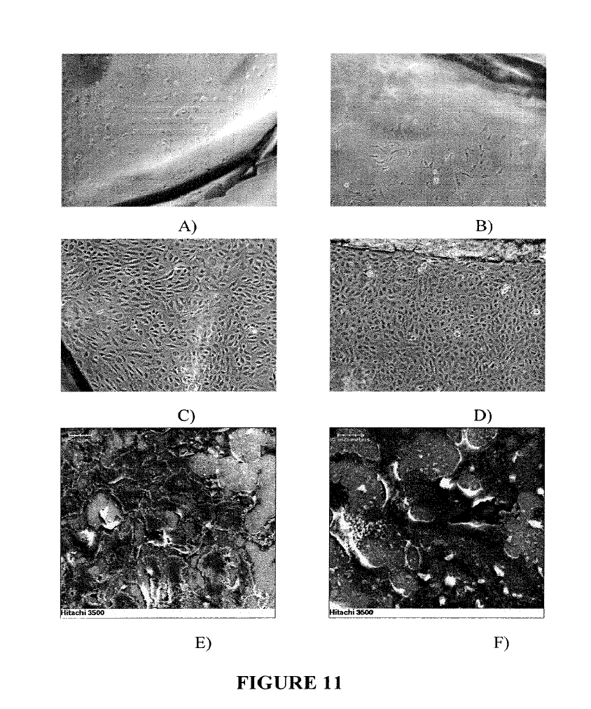

The in vitro biocompatibility of POC was evaluated in order to investigate the potential application in tissue engineering, especially for soft tissue engineering such as vascular graft, ligament, bladder, and cartilage. Human smooth muscle cells and endothelial cells are chosen as model cells. FIGS. 9 and 11 show the morphology of both cell types on POC films at different culture times. The results indicate that POC is a good substrate for supporting the both cells attachment. Both cells grow promptly and achieve confluence on POC.

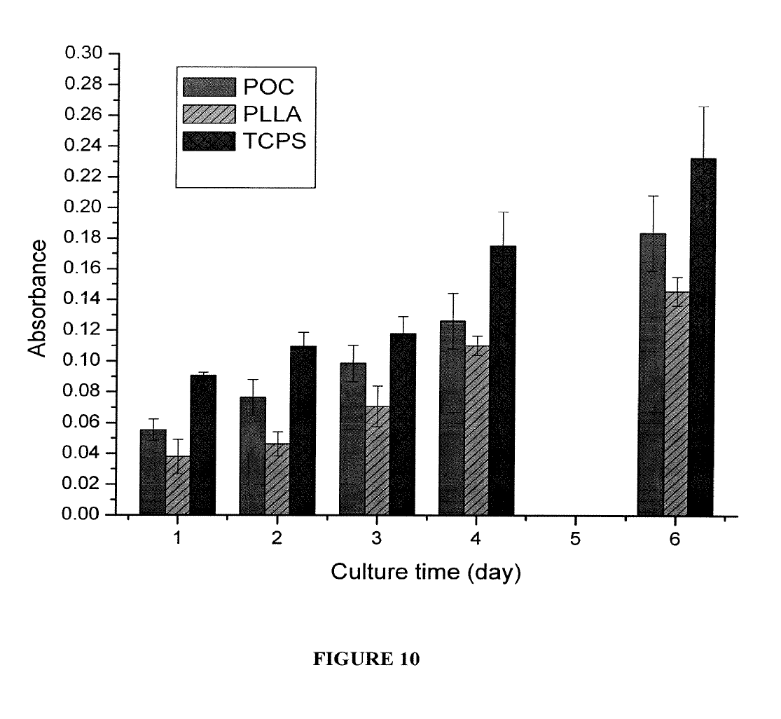

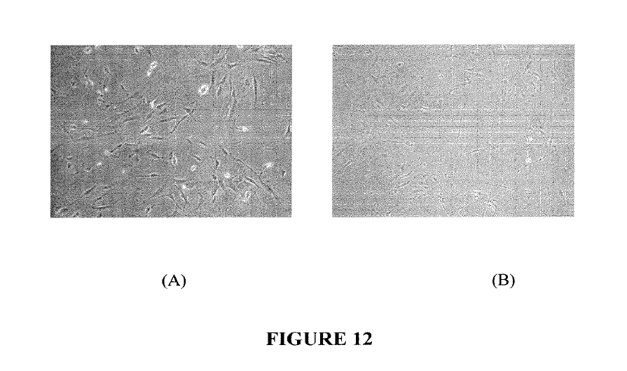

Cell attachment and growth are also observed on PDC (FIG. 12). MTT assays (an indicator of cell viability) also indicate that POC is a better substrate for cell growth than PLLA (FIG. 10). Synthetic materials have attracted many interests as small diameter grafts. Normally, the synthetic grafts have not produced acceptable results because of rapid thrombotic buildup in the vessel lumen [13]. Researchers have been attempting to improve graft performance by adding an endothelial lining and thus better mimicking the vessels in the body [14,15]. Failure of grafts was associated with subintimal hyperplasia and a thrombotic surface, possibly resulting in part from lack of a confluent layer of endothelial cells on the graft lumen. Many methods have been developed for improving the endothelial cell attachment and growth such as immobilizing cell adhesion peptides (GREDVY) on polymer surfaces [16], plasma modification using radio frequency glow discharge [17] and so on. Endothelial cells adherence can be dramatically increased when the grafts are coated with extracellular matrix, plasma or fibronectin. Unfortunately for graft compatibility, coating with fibronectin increases not only the adhesion of endothelial cells to those surfaces, but of platelets as well [18]. Optimal adherence has been reported for gas plasma- treated surfaces with hydrophilicity in the range of 40-60° by Dekker [19] and van Wachem [20]. This effect was attributed to specific protein adsorption favorable for adhesion, spreading, and proliferation of endothelial cells, and improved deposition of endothelial matrix proteins. For POC, the hydrophilicity is in the above range, which may help the adsorption of glycoproteins on the polymer surface. The surface-enriched polar groups such as carboxyl and hydroxyl may facilitate the cell attachment and growth [21,22]. No additional pre-treatments are needed and the endothelial cells confluence on POC films can be achieved in a short time.

### Example 4

### Synthesis of Poly (1,6-Hexanediol-Co-Citric Acid) (PHC)

In a typical experiment, 19.212 g citric acid and 11.817 g 1,6-hexanediol were added to a 250 ml three-neck round-bottom flask, fitted with an inlet adapter and an outlet adapter. The mixture was melted within 15 min by stirring at 160-165° C. in a silicon oil bath, and then the temperature of the system was lowered to 120° C. The mixture was stirred for half an hour at 120° C. to get the pre-polymer. Nitrogen was vented throughout the above procedures. The pre-polymer was post-polymerized at 60° C., 80° C. or 120° C. with and without vacuum for a predetermined time from one day to 3 weeks, depending on the temperature, to achieve the Poly (1,6-hexanediol-co-citric acid). Nitrogen was introduced into the reaction system before the polymer was taken out from reaction system.

### Example 5

### Synthesis of Poly (1,10-Decanediol-Co-Citric Acid) (PDC)

In a typical experiment, 19.212 g citric acid and 17.428 g 1,10-decanediol were added to a 250 ml three-neck round-bottom flask, fitted with an inlet adapter and an outlet adapter. The mixture was melted within 15 min by stirring at 160-165° C. in silicon oil bath, and then the temperature of the system was lowered to 120° C. The mixture was stirred for half an hour at 120° C. to get the pre-polymer. Nitrogen was vented throughout the above procedures. The pre-polymer was post-polymerized at 60° C., 80° C. or 120° C. with and without vacuum for predetermined time from one day to 3 weeks depending on the temperature to achieve the Poly (1,10-decanediol-co-citric acid). Nitrogen was introduced into the reaction system before the polymer was taken out from reaction system.

### Example 6

### Synthesis of Poly (1,12-Dodecanediol-Co-Citric Acid) PDDC

In a typical experiment, 19.212 g citric acid and 20.234 g 1,12-dodecanediol were added to a 250 ml three-neck round-bottom flask, fitted with an inlet adapter and an outlet adapter. The mixture was melted within 15 min by stirring at 160-165° C. in silicon oil bath, and then the temperature of the system was lowered to 120° C. The mixture was stirred for half an hour at 120° C. to get the pre-polymer. Nitrogen was vented throughout the above procedures. The pre-polymer was post-polymerized at 60° C., 80° C. or 120° C. with and without vacuum for predetermined time from one day to 3 weeks depending on the temperature to achieve the Poly (1,12-dodecanediol-co-citric acid). Nitrogen was introduced into the reaction system before the polymer was taken out from reaction system.

### Example 7

### Synthesis of Poly(1,8-Octanediol-Co-Citric Acid-Co-Glycerol)

In a typical experiment (Poly(1,8-octanediol-co-citric acid-co-1% glycerol), 23.0544 g citric acid, 16.5154 g 1,8-octanediol and 0.2167 g glycerol were added to a 250 ml three-neck round-bottom flask, fitted with an inlet adapter and an outlet adapter. The mixture was melted within 15 min by stirring at 160-165° C. in silicon oil bath, and then the temperature of the system was lowered to 120° C. The mixture was stirred for another hour at 140° C. to get the pre-polymer. Nitrogen was vented throughout the above procedures. The pre-polymer was post- polymerized at 60° C., 80° C. or 120° C. with and without vacuum for predetermined time from one day to 3 weeks depending on the temperature to achieve the Poly (1,8-octanediol-co-citric acid-co-1% glycerol). Nitrogen was introduced into the reaction system before the polymer was taken out from reaction system.

### Example 8

### Synthesis of Poly(1,8-Octanediol-Citric Acid-Co-Polyethylene Oxide)

In a typical experiment, 38.424 g citric acid, 14.623 g 1,8-octanediol and 40 g polyethylene oxide with molecular weight 400 (PEO400)(100 g PEO1000 and 200 g PEO2000 respectively) (molar ratio: citric acid/1,8-octanediol /PEO400=1/0.5/0.5) were added to a 250 ml or 500 ml three-neck round-bottom flask, fitted with an inlet adapter and an outlet adapter. The mixture was melted within 15 min by stirring at 160-165° C. in silicon oil bath, and then the temperature of the system was lowered to 135° C. The mixture was stirred for 2 hours at 135° C. to get the pre-polymer. Nitrogen was vented throughout the above procedures. The pre-polymer was post-polymerized at 120° C. under vacuum for predetermined time from one day to 3 days to achieve the Poly(1,8-octanediol-citric acid-co-polyethylene oxide). Nitrogen was introduced into the reaction system before the polymer was taken out from reaction system. The molar ratios can be altered to achieve a series of polymers with different properties.

### Example 9

### Synthesis of Poly(1,12-Dodecanediol-Citric Acid-Co-Polyethylene Oxide)

In a typical experiment, 38.424 g citric acid, 20.234 g 1,12-dodecanediol and 40 g polyethylene oxide with molecular weight 400 (PEO400)(100 g PEO1000 and 200 g PEO2000 respectively) (molar ratio: citric acid/1,8-octanediol /PEO400=1/0.5/0.5) were added to a 250 ml or 500 ml three-neck round-bottom flask, fitted with an inlet adapter and an outlet adapter. The mixture was melted within 15 min by stirring at 160-165° C. in silicon oil bath, and then the temperature of the system was lowered to 120° C. The mixture was stirred for half an hour at 120° C. to get the pre-polymer. Nitrogen was vented throughout the above procedures. The pre-polymer was post-polymerized at 120° C. under vacuum for predetermined time from one day to 3 days to achieve the Poly(1,12-dodecanediol-citric acid-co-polyethylene oxide). Nitrogen was introduced into the reaction system before the polymer was taken out from reaction system. The molar ratios can be altered to achieve a series of polymers with different properties.

### Example 10

### Synthesis of Poly(1,8-Octanediol-Citric Acid-Co—N-Methyldiethanoamine) POCM

In a typical experiment, 38.424 g citric acid, 26.321 g 1,8-octanediol and 2.3832 g N-methyldiethanoamine (MDEA) (molar ratio: citric acid/1,8-octanediol/MDEA=1/0.90/0.10) were added to a 250 ml or 500 ml three-neck round-bottom flask, fitted with an inlet adapter and an outlet adapter. The mixture was melted within 15 min by stirring at 160-165° C. in silicon oil bath, and then the temperature of the system was lowered to 120° C. The mixture was stirred for half an hour at 120° C. to get the pre-polymer. Nitrogen was vented throughout the above procedures. The pre-polymer was post- polymerized at 80° C. for 6 hours, 120° C. for 4 hours without vacuum and then 120° C. for 14 hours under vacuum to achieve the Poly(1,8-octanediol-citric acid-co-N-methyldiethanoamine). Nitrogen was introduced into the reaction system before the polymer was taken out from reaction system. The molar ratios can be altered to citric acid/1,8-octanediol /MDEA=1/0.95/0.05.

### Example 11

### Synthesis of Poly(1,12-Dodecanediol-Citric Acid-co-N-Methyldiethanoamine) PDDCM

In a typical experiment, 38.424 g citric acid, 36.421 g 1,12-dodecanediol and 2.3832 g N-methyldiethanoamine (MDEA) (molar ratio: citric acid/1,8-octanediol/MDEA=1/0.90/0.10) were added to a 250 ml or 500 ml three-neck round-bottom flask, fitted with an inlet adapter and an outlet adapter. The mixture was melted within 15 min by stirring at 160-165° C. in a silicon oil bath, and then the temperature of the system was lowered to 120° C. The mixture was stirred for half an hour at 120° C. to get the pre-polymer. Nitrogen was vented throughout the above procedures. The pre-polymer was post-polymerized at 80° C. for 6 hours, 120° C. for 4 hours without vacuum and then 120° C. for 14 hours under vacuum to achieve the Poly(1,12-dodecanediol-citric acid-co-N-methyldiethanoamine). Nitrogen was introduced into the reaction system before the polymer was taken out from reaction system. The molar ratios can be altered to citric acid/1,12-dodecanediol/MDEA=1/0.95/0.05.

### Example 12

### Calcium Modification Of Different Polymers

In a typical experiment, POC and PDDC films or scaffolds were immersed in a 0.1 M CaCl₂ solution for 1 week, rinsed in mini-Q water and then freeze-dried. The dry samples were stored in a desiccator before use. In order to evaluate calcium modification on mechanical properties of POC and PDDC, POC and PDDC was tested under different treating conditions.

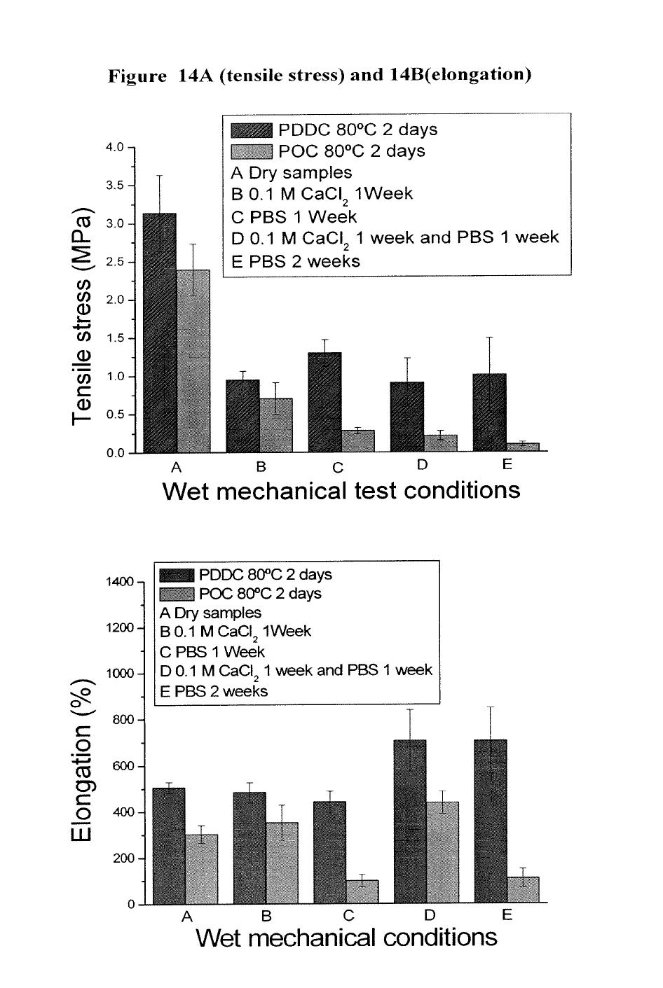

Mechanical test results show that calcium under wet conditions, calcium treatment (1 week) may help to maintain appropriate tensile stress for POC compared to PBS 1 week of treatment with phosphate buffered saline (PBS). Calcium treatment has dramatic effects on elongation for POC. After 1 week of calcium treatment, POC can maintain a similar elongation rate compared to 1 week of PBS (phosphate buffer solution) 1 week treatment. Even after 1 more week of PBS treatment following 1 week of calcium treatment, the height elongation rate of POC can be still maintained. Since PDDC is more hydrophobic than POC, the effects of calcium treatment on tensile stress and elongation of PDDC is less than that on POC. This results show that calcium ions chelated by unreacted carboxyl group of POC and PDDC synthesized under mild condition (80° C. 2 days) act as crosslinkers to help to maintain the elasticity and appropriate strength of polymers (FIG. 14).

### Example 13

### Synthesis of POC-Hydroxyapatite (HA) Composite

In a typical experiment, 19.212 g citric acid and 14.623 g Octanediol (molar ratio 1:1) of Citric Acid and 1,8-octanediol was reacted in a 250 ml three-neck round-bottom flask at 165° C., forming a pre-polymer solution. Specific amount of HA was then added to reaction vessel while stirring with a mechanical stirrer. Acetone was then added until solution liquefied into a slurry state. Solution was then cast into a Teflon mold and set in a vacuum oven at 120° C. for 2-4 hours or until the acetone was purged. Film was then incubated without vacuum at 120° C. overnight allowing the solution to set. Film could then be post polymerized for various durations depending upon the desired properties. Mechanical tests on POC-HA (40 wt %) shows tensile stress is as high as 10.13±0.57 MPa and elongation is 47.78±3.00. Specimen recovery completely after pulling by mechanical tester.

### Example 14

### Comparison of the Properties of Different Polymers

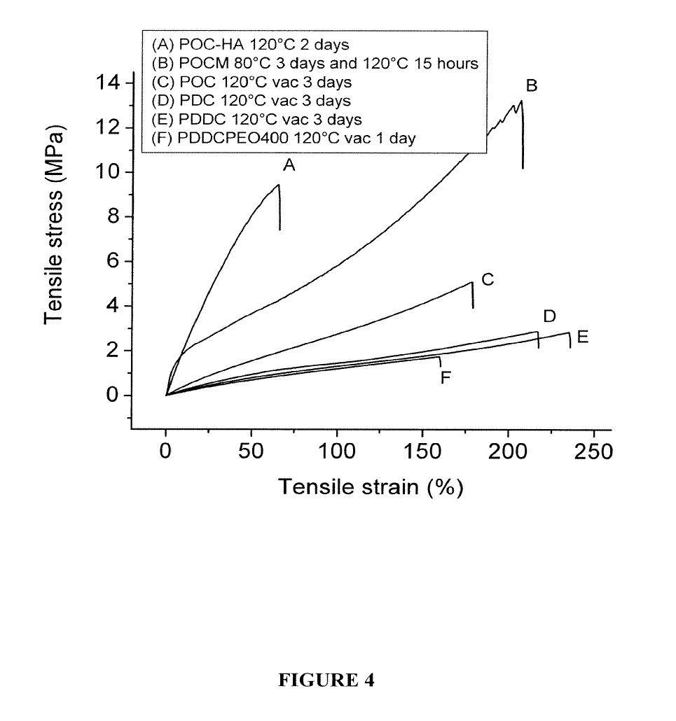

FIG. 4 shows that the mechanical properties of the polymer can be modulated by choosing different diol monomers. The maximum elongation ratio for the polymer at break can reach 265±10.5% similar to that of arteries and vein (up to 260%) [10]. The minimum tensile Young's modulus can reach 1.4±0.2 MPa. The Young's modulus is between those of ligament (KPa scale) [11] and tendon (GPa scale) [12].

Similar to the vulcanized rubber, POC, PDC, and PDDC are thermoset elastomers. In general, thermoset polymers can not be dissolved in common solvents which adds to the difficulty in making the polymer into a scaffold for tissue engineering applications. The present application describes a method to fabricate porous and non-porous scaffolds which makes it possible to be used in tissue engineering utilizing the solubility of the pre-polymer in some solvent such as dioxane, acetone, 1,3-dioxlane, ethanol, N,N-dimethylformamide. Therefore, this family of polymers is a potential elastomer in tissue engineering especially in soft tissue engineering.

### Example 15

### Further Characterization of Solid Polymeric Materials

This example is directed to the extent of cross-linking of the polymeric materials. Current methods to determine the molecular weight of a polymer include osmotic pressure, light scattering, ultracentrifugation, solution viscosity, and gel permeation chromatography measurements. All of these methods normally require a polymer that can be dissolved in specific solvents [24] Crosslinked polymers can not be dissolved in a solvent and their molecular weight is considered to be infinite. However, a useful parameter to characterize cross-linked polymers is molecular weight between cross-links (Mc), which can give a measure of the degree of cross-linking and therefore some insight into mechanical properties. According the theory of rubber elasticity, molecular weight between crosslinks can be calculated using Equation (1) under some assumptions[25]:

$$n = \frac{{E}_{0}}{3 RT} = \frac{ρ}{{M}_{c}}$$

where n represents the number of active network chain segments per unit volume; Mc represents the molecular weight between cross-links (mol/m³); E₀ represents Young's modulus (Pa); R is the universal gas constant (8.3144 Jmol-1K-1); T is the absolute temperature (K); ρ is the elastomer density (g/m.sup.3) as measured via volume method. .sup.[26] From Equation (1), molecular weight between crosslinks can only be obtained after mechanical tests and polymer density measurements. Another method for determining molecular weight between crosslinks for a crosslinked polymer is by swelling the polymer[27] Using the swelling method, molecular weight between crosslinks can be calculated by Equation (2).

$$\frac{1}{{M}_{c}} = \frac{2}{{M}_{n}} - \frac{\frac{υ}{{V}_{1}} [ ln ( 1 - {υ}_{2 , s} ) + {υ}_{2 , s} + {χ}_{1} {υ}_{2 , s}^{2} ]}{{υ}_{2 , s}^{1 / 3} - \frac{{υ}_{2 , s}}{2}}$$

where Mc is the number average molecular weight of the linear polymer chain between cross-links, ν is the specific volume of the polymer, V₁ is the molar volume of the swelling agent and ×₁ is the Flory-Huggins polymer-solvent interaction parameter. ν_{2,s} is the equilibrium polymer volume fraction which can be calculated from a series of weight measurements.

### Example 16

### Novel Biphasic Scaffold Design for Blood Vessel Tissue Engineering

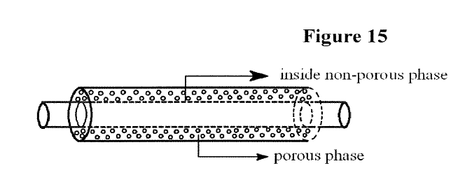

Biphasic scaffolds consist of outside porous phase and inside non-porous phase as depicted in the schematic drawing shown in FIG. 15. The non-porous phase is expected to provide a continuous surface for EC adhesion and spreading, mechanical strength, and elasticity to the scaffold. The porous phase will facilitate the 3-D growth of smooth muscle cells. Biphasic scaffolds were fabricated via following procedures. Briefly, glass rods (˜3 mm diameter) were coated with the pre-polymer solution and air dried to allow for solvent evaporation. Wall thickness of the tubes can be controlled by the number of coatings and the percent pre-polymer in the solution. The pre-coated pre-polymer was partially post-polymerized under 60° C. for 24 hr; the pre-polymer-coated glass rod is then inserted concentrically in a tubular mold that contains a salt/pre-polymer slurry. The pre-polymer/outer-mold/glass rod system is then placed in an oven for further post-polymerization. After salt-leaching [4], the biphasic scaffold was then de-molded from the glass rod and freeze dried. The resulting biphasic scaffold was stored in a desiccator before use. The same materials or different materials from the above family of elastomers can be utilized for both phases of the scaffold. Other biomedical materials widely used in current research and clinical application such as polylactide (PLA), polycaprolactone (PCL), poly(lactide-co-glycolide) (PLGA) may also be utilized for this novel scaffold design.

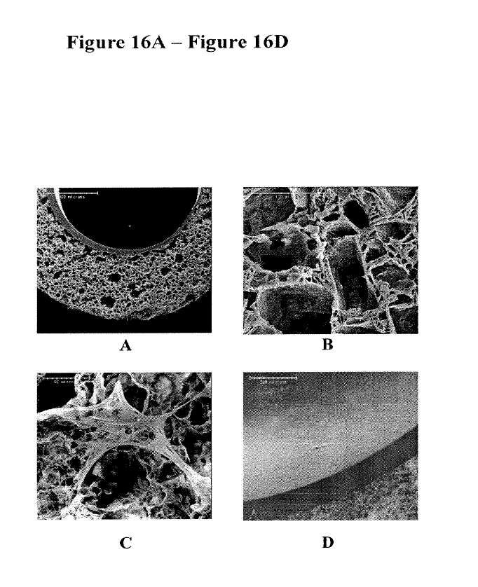

The thickness, degradation, and mechanical properties of inside non-porous phase can be well controlled by choosing various pre-polymers of this family of elastomers, pre-polymer concentration, coating times and post-polymerization conditions (burst pressure can be as high as 2800 mmHg). The degradable porous phase and non-porous phases are integrated since they are formed in-situ via post-polymerization. The cell culture experiments shown in FIG. 16 confirm that both HAEC and HASMC can attach and grow well in biphasic scaffolds. The results suggest that a biphasic scaffold design based on poly(diol-co-citrate) is a viable strategy towards the engineering of small diameter blood vessels.

### Example 17

### Materials and Methods Employed for Polymer Characterization

In addition to the materials and methods described above, the following materials and methods also are exemplary of the studies performed herein.

### Polymer Synthesis

Preparation of poly(1,8-Octanediol-co-citric acid) (POC) films: [23] All chemicals were purchased from Sigma-Aldrich (Milwaukee, Wis.). Equimolar amounts of citric acid and 1,8-octanediol were added to a 250 ml three-neck round-bottom flask, fitted with an inlet and outlet adapter. The mixture was melted under a flow of nitrogen gas by stirring at 160° C.-165° C. in a silicon oil bath, and then the temperature of the system was lowered to 140° C. The mixture was stirred for another hour at 140° C. to create the pre-polymer solution. The pre-polymer was cast in glass dishes and post-polymerized at 80° C., 120° C. or 140° C. under vacuum (2 Pa) or no vacuum for times ranging from 1 day to 2 weeks to create POC films with various degrees of cross-linking.

### Mechanical Tests

Tensile tests were conducted according to ASTM D412a on an Instron 5544 mechanical tester equipped with 500 N load cell (Instron Canton, Mass.). Briefly, a dog-bone-shaped sample (26×4×1.5 mm, Length.times.Width.times.Thickness) was pulled at a rate of 500 mm/min. Values were converted to stress-strain and a Young's modulus was calculated. 4-6 samples were measured and averaged.

### Molecular Weight Between Crosslinks Measurements

The molecular weight between crosslinks of POC was calculated using Equation (1).

### Swelling Studies

Polymers were cut into rectangular strip and the initial length, width and thickness measured with calipers. The polymers were then swollen in DMSO at 37° C. overnight to achieve equilibrium swelling. The equilibrium length, width, and thickness were measured to determine the change in volume upon swelling.

Results and DiscussionMechanical Tests and Molecular Weight Measurements of POC post-polymerization conditions and the resulting polymer films were subjected to mechanical tensile tests and molecular weight between crosslinks measurements[25] The results in Table 1 indicate that increased crosslinking temperatures and time increase the tensile stress, Young's modulus and the number of active network chain segment per unit volume (crosslinking density) while decreasing the molecular weight between crosslinks. Therefore, the mechanical properties of POC can be well controlled by controlling polymer network structures via post-polymerization under different conditions.

TABLE 1
Mechanical properties, the number of active network chain segment per unit
volume (crosslinking density: n) and molecular weight between crosslinks (Mc) of POC
synthesized under different conditions

| | | Young's | Tensile | | |
|---|---|---|---|---|---|
| | Polymerization | Modulus | Stress | n | Mc |
| POC | condition | (MPa) | (MPa) | (mol/m³) | (g/mol) |
| LS1 | 80° C., no vacuum, 2 | 1.38 ± 0.21 | 1.64 ± 0.05 | 182.59 ± 27.78 | 6874 ± 148 |
| | days | | | | |
| LS2 | 80° C., high vacuum, 2 | 1.72 ± 0.45 | 1.90 ± 0.22 | 227.58 ± 59.54 | 5445 ± 116 |
| | days | | | | |
| LS3 | 120° C., high vacuum, 1 | 2.84 ± 0.12 | 3.62 ± 0.32 | 375.77 ± 15.88 | 3301 ± 218 |
| | day | | | | |
| LS4 | 120° C., high vacuum, 2 | 3.13 ± 0.27 | 3.66 ± 0.61 | 414.14 ± 35.72 | 2971 ± 76 |
| | days | | | | |
| LS5 | 120° C., high vacuum, 3 | 4.69 ± 0.48 | 5.34 ± 0.66 | 620.68 ± 63.51 | 1857 ± 81 |
| | days | | | | |
| LS6 | 140° C., high vacuum, 2 | 6.07 ± 0.52 | 5.73 ± 1.39 | 803.14 ± 68.80 | 1516 ± 269 |
| | days | | | | |
| LS7 | 80° C., no vacuum, 5 | 2.21 ± 0.17 | 3.90 ± 0.60 | 292.41 ± 22.49 | 4326 ± 68 |
| | days | | | | |
| LS8 | 80° C., no vacuum, 14 | 2.24 ± 0.09 | 2.55 ± 0.21 | 296.38 ± 11.91 | 4265 ± 33 |
| | days | | | | |

### Example 18

### Preparation and Characterization of a Nanoporous Biodegradable Thermoset Elastomer

Materials: All chemicals were purchased from Sigma-Aldrich (Milwaukee, Wis.): Polyethylene glycol dimethyl ether [Mₙ 500], 1,8 octanediol (98%) and citric acid (99.5%).

Sample preparation: POC pre-polymer was synthesized as follows. Briefly, equimolar amounts of citric acid and 1,8 octanediol were melted at 160-165° C. under a flow of nitrogen gas while stirring. The temperature of the system was subsequently lowered to 140° C. for 30 min under stirring to create a POC pre-polymer. The pre-POC was purified by precipitation in water and then lyophilized. To create control non-porous POC films, the pre-polymer solution was post-polymerized at 80° C. for 4 days without vacuum. For nanoporous POC, a 75/25 (w/w) PEGDM/POC pre-polymer solution was blended and subsequently incubated at 120° C. for 3 days at 2 Pa to cross-link the POC pre-polymer. The PEGDM was removed by leaching the polymer films in deionized water (MilliQ) with sonication. Samples were dried using a Polaron critical point dryer.

A leaching time-course evaluation was used to verify that most of the PEGDM had been removed within 3-7 days of polymer leaching. For porous POC scaffolds with micron scale porosities as comparison in future drug delivery studies, NaCl particles were sifted to 100-250 microns and mixed with POC pre-polymer for a 90/10 (w/w) salt/POC pre-polymer mixture. Salt was similarly leached in deionized water with sonication and lyophilized. AgNO₃ was used to verify the absence of NaCl in the polymer leachate solution.

Evaluation of PEGDM stability and non-reactivity during POC polycondensation reaction conditions: Fourier transform infrared (FTIR) spectra were obtained at room temperature using a FTS40 Fourier transform infrared spectrometer (BioRad Hercules, Calif.). Samples were prepared by a solution-casting technique over a KBr crystal (samples included a 30% by wt. control pre-polymer POC diluted in ethanol, 100% PEGDM solution, pre-polymer POC to PEGDM 1:5 wt. to wt. ratio solution and the same solution incubated at 120° C. at 2 Pa for 3 days).

Characterization of the morphology of POC and nanoporous POC: For SEM, control POC films and nanoporous POC samples were sputter coated with palladium/gold and examined using a LEO Gemini 1525 SEM.

Characterization of the polymer pore size distribution: Mercury intrusion porosimetry was assessed using a Micromeritics AutoPore IV 9500 V1.06 to determine pore size distribution, pore volume and density of POC scaffolds. A relationship between applied external pressure and intruded volume of mercury into the pores of the sample material is provided by the Washburn equation [54]:

$$Δ P = \frac{2 σ cos θ}{R}$$

The value ΔP is the applied pressure, R is the pore radius, σ is the surface tension and θ is the contact angle. A mercury contact angle of 130° and an interfacial tension of mercury of 485 dynes/cm was used. This technique can evaluate pore size distributions from 3 nm-360 μm.

Characterization of the mechanical properties of POC and nanoporous POC: Tensile mechanical tests were conducted according to ASTM D412a on an Instron 5544 mechanical tester equipped with 500 N load cell (Instron, Canton, Mass.). Briefly, the dog-bone-shaped sample (26×4×1.5 mm. length×width×thickness) was pulled at a rate of 500 mm/min. Values were converted to stress-strain and a Young's modulus was calculated from the initial sloping region of the stress-strain curve. The cross-link density (n) was calculated according to the theory of rubber elasticity using the following equation [14-15]:

$$n = \frac{{E}_{0}}{3 RT} = \frac{ρ}{{M}_{c}}$$

The value n represents the number of active network chains segments per unit volume (mol/m³), M_{c} represents the molecular weight between cross-links (g/mol), E₀ represents Young's modulus (Pa), R is the universal gas constant (8.31144 J/mol K), T is the absolute temperature (K) and ρ is the elastomer density (g/m³). Elastomer density for non-porous POC films have been calculated based on Archimedes' principle in a previous study [45]. For porous POC samples prepared by methods described above, elastomer density was measured using mercury intrusion porosimetry.

### Characterization of the In Vitro Degradation of Nanoporous POC:

Disk-shaped specimens (10 mm in diameter, about 1-1.5 mm thickness) were placed in a tube containing 10 ml phosphate buffer saline (PBS) (pH 7.4) or 25 mM NaOH to rapidly obtain relative degradation rates among different samples. Specimens were incubated at 37° C. in PBS or NaOH solution for pre-determined times, respectively. After incubation, samples were washed with water and lyophilized. A total of five to six samples were performed for the degradation test. Mass loss was calculated by comparing the initial mass (W₀) with the mass measured at a given time point Wₜ as shown in the equation below:

$$Mass loss ( \% ) = \frac{{W}_{0} - {W}_{t}}{{W}_{0}}$$

### Results

### Pore Size Distribution, Porosity and Surface Area.

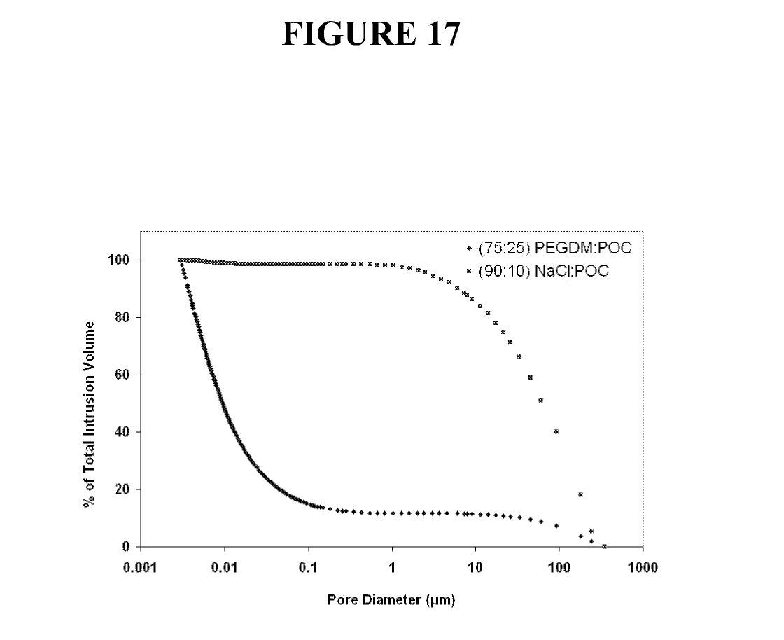

The median pore diameter for nanoporous POC scaffolds was 9.5 nm compared to 63.5 μm for the control salt leached POC scaffolds. Total pore area (m²/g) was 35.28 and 29.33 for the nanoporous POC and control porous POC scaffolds, respectively (See Table 2). The average porosity for the dried nanoporous POC was 8.4% compared to an average porosity of 79.1% for the salt leached scaffolds. FIG. 17 shows a plot of the percent of total mercury intrusion volume compared to the pore size diameter (μm).

TABLE 2
Mechanical tests, cross-linking characterization, and pore size distribution
measurements.

| | Young's | | | Median | | Surface |
|---|---|---|---|---|---|---|
| | modulus | Elongation at | | pore | Porosity | area |
| Sample | (MPa) | break (%) | M_{c} (g/mol) | diameter | (%) | (m²/g) |
| POC | 1.49 ± 0.10 | 173.69 ± 13.62 | 6200 ± 389 | NA | NA | NA |
| 90/10 (w/w) | NA | NA | NA | 63.5 μm | 79.1 | 29.33 |
| NaCl/POC | | | | | | |
| 75/25 (w/w) | 0.11 ± 0.02 | 404.71 ± 17.61 | 83987 ± 12921 | 9.5 nm | 9.5 | 35.28 |
| PEGDM/POC | | | | | | |

### Mechanical Properties.

The Young's moduli for control non-porous POC films and the nanoporous POC were 1.49±0.10 MPa and 0.11±0.02 MPa, respectively. The nanoporous POC was highly distensible with a 404.71±17.61% elongation at break compared to 173.69±13.62% elongation at break for control samples (n=6,+SD) (See FIG. 2). Both the nanoporous POC and control POC films did not undergo permanent plastic deformation after breakage. The average molecular weight between cross-links (g/mol) for nanoporous POC was 83,987±12,921 and 6,200±389 for control POC films.

### Degradation.

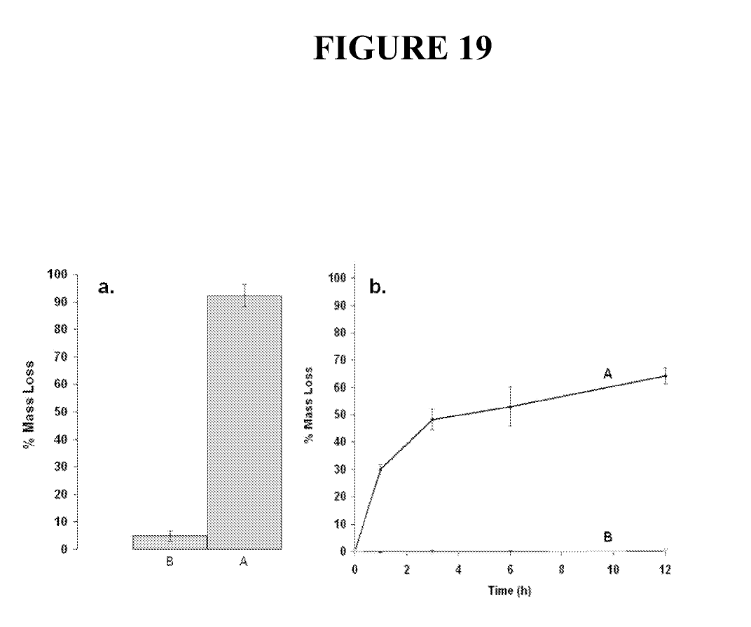

The results of degradation studies in PBS show almost complete degradation of nanoporous POC after 1 week incubation with a percent mass loss of 92.4±4.0 whereas control non-porous POC films showed only minor degradation with a percent mass loss of 4.8±1.9 (FIG. 19a). Accelerated degradation studies in NaOH revealed a percent mass loss of 64.2±2.9 and 0.5±0.5 for nanoporous POC and control POC films respectively after 12 hours of incubation (FIG. 19b).

### Morphology Using SEM:

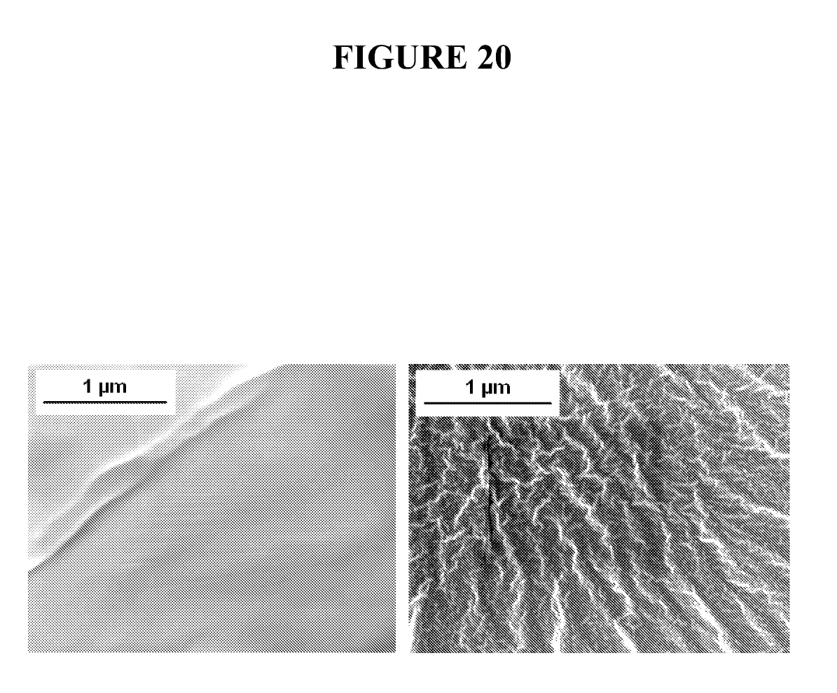

Samples analyzed via SEM consisted of: 1) control POC film containing 50% wt. PEGDM and 2) POC film containing 50% wt. PEGDM that was incubated in multiple changes of acetone for approximately 24 hours (to remove PEGDM) and critically point dried. FIGS. 20a and 20b are representative images taken from the non leached sample and leached samples respectively. The non leached sample which contains both polymerized POC and the PEGDM porogen shows a relatively flat surface topology in contrast to the PEGDM leached sample which shows a fibrous structural surface characteristic of the void space left behind by the leached PEGDM phase in the cross-linked nanoporous POC network.

### FTIR Spectra:

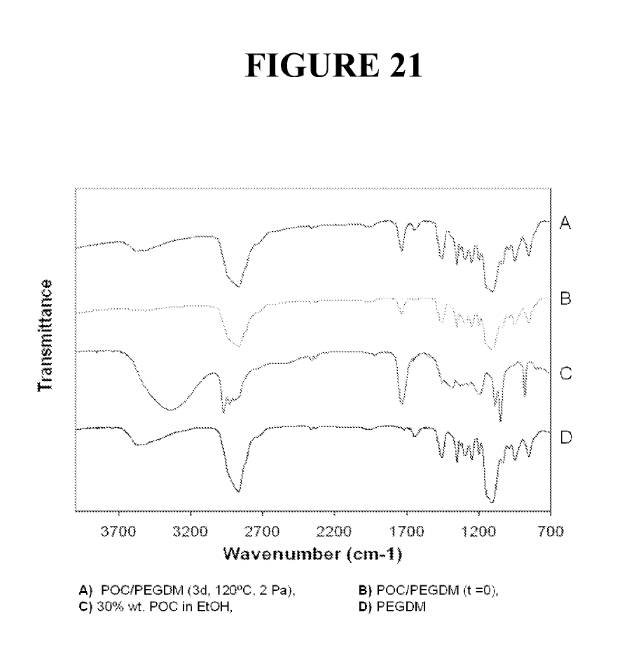

The FTIR analysis of the POC, PEGDM and PEGDM/POC mixtures before and after polymerization are shown in FIG. 21. The inverted peaks within 1690-1750 cm⁻¹ were assigned to carbonyl groups (C═O). The inverted peak centered at 2870 cm⁻¹ was assigned to the methyl group of PEGDM and the inverted peak centered at 1120 cm⁻¹ was assigned to the ether bonds of PEGDM. The distinct methyl groups and ether groups are only shown in the IR spectra for samples containing PEGDM and remained in polymerized POC samples. The ether bonds remained stable even after incubations at 120° C. for 3 days.

### Solubility Parameter Calculation:

The solubility parameter for pre-polymer POC and PEGDM was calculated to measure the interaction and solubility effects of these two systems. Generally for two chemical components to be completely miscible with one another, the solubility parameter, Δδ, should be <3.7 (J^{1/2}/cm^{3/2}) [16]. The solubility parameter is equal to the sum of dispersion, polar and hydrogen bonding contributions (see equation below) [58-59].

δ²=δ_{d}²+δₚ²+δₕ²

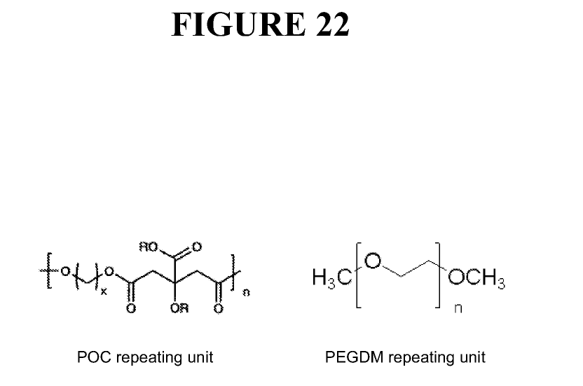

In order to form nanoporous polymer networks during polymerization, the pre-polymer POC should generally be completely miscible with the PEGDM porogen without macro and microscopic phase separation. For the solubility parameter calculations, it was assumed that the POC repeating functional group “R” included carbon bond (carboxyl group) and the other functional group “R” included a hydrogen bond (hydroxyl group) (Refer to FIG. 22). POC prepolymer has a molecular weight between 1,000-2,000 g/mol [44]. The POC prepolymer density was assumed to be approximately the same as cross-linked POC. The solubility parameter for pre-polymer POC was calculated to be, δ=22.28 (J^{1/2}/cm^{3/2}) and δ=19.90 (J^{1/2}/cm^{3/2}) for PEGDM. The difference in the two solubility parameters of Δδ=2.38 satisfies the condition for <3.7 (J^{1/2}/cm^{3/2}).

### FTIR Analysis

FTIR analysis was performed to confirm the stability of PEGDM under the POC polymerization conditions. PEGDM is an ideal aprotic polar solvent and is miscible with pre-polymer POC solutions in ethanol. Since PEGDM has no additional functional groups, it is relatively chemically inert and stable at elevated temperatures [48], which makes it suitable as a porogen during POC polymerization.

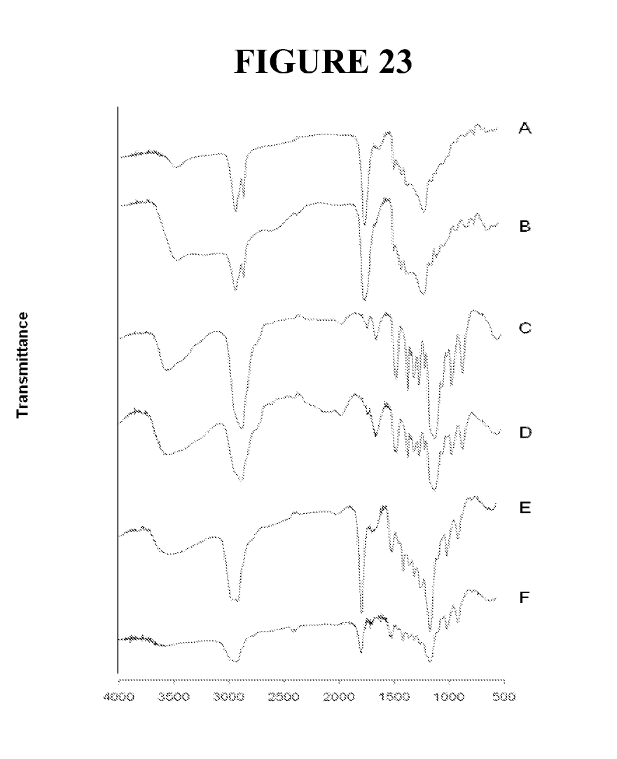

Fourier transform infrared (FTIR) spectra were obtained using a FTS40 Fourier transform infared spectrometer (BioRad, Hercules, Calif.). Polymer samples were prepared by a solution-casting technique by drop-wise adding 5 microliters of sample solution onto a KBr crystal slide. For t=0 h incubation time points, samples were allowed to air dry overnight under vacuum. For polymerized samples, after air drying, samples were placed in a 120° C. oven at 2 Pa for t=24 hours. FIG. 23 shows representative FTIR spectra for pre-polymer POC, PEGDM and sample mixtures of POC and PEGDM before and after polymerization. Peaks centered at 1690-1750 cm⁻¹ comprising the carbonyl groups and peaks centered at 2931 cm⁻¹ comprising methylene groups of POC polymer chains are present in samples A, B, E and F which all contain either pre-polymer POC or polymerized POC. Peaks centered at 2895 cm⁻¹ comprising methyl groups and peaks centered at 1120 cm⁻¹ comprising ether bonds of PEGDM are still present in all samples containing PEGDM (C,D,E,F). Similar FTIR spectra for samples D and C represent PEGDM before and after incubation at 120° C. reflecting its thermal stability under the POC polymerization reaction conditions.

### SEM Evaluation of Polymer Pore Structure

In order to confirm the technique used to create nanoporous polymer networks using PEGDM as the porogen during POC polymerization, SEM was used to get an idea of the pore structure formation and pore size.

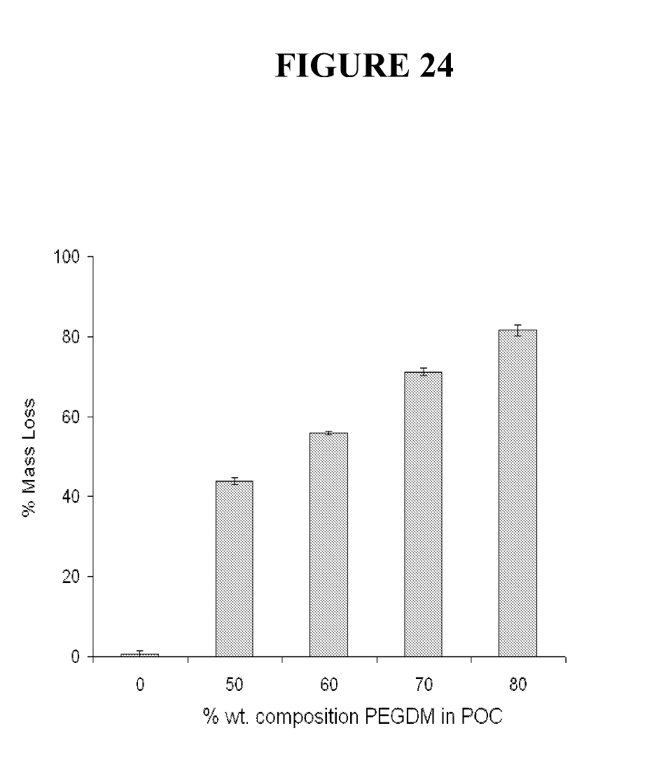

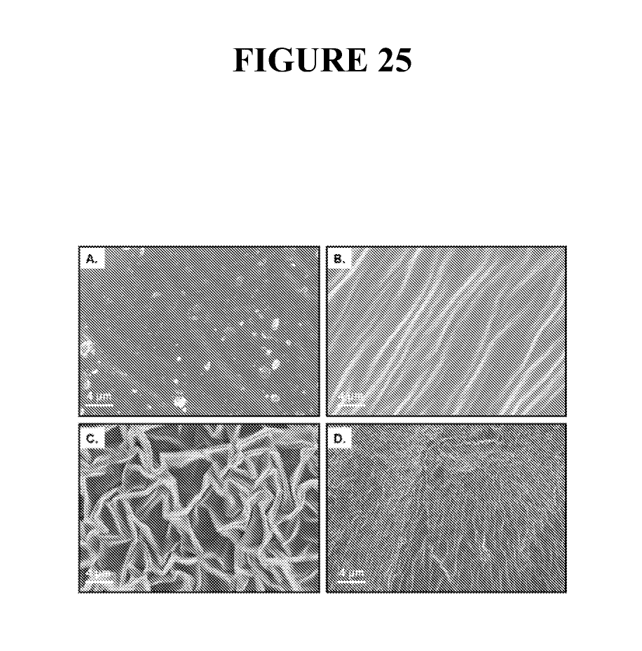

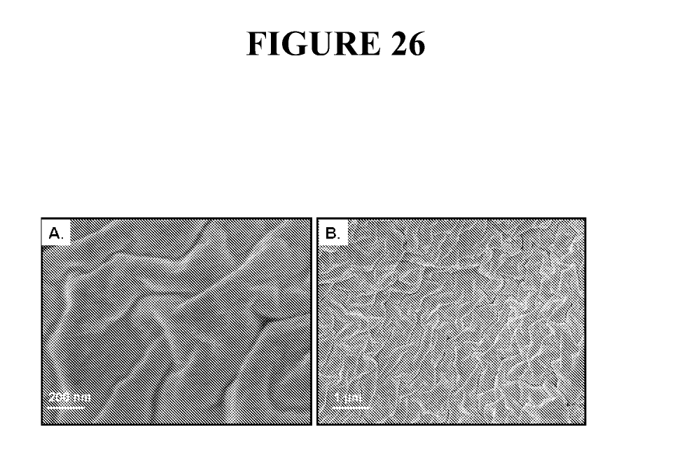

SEM was used to confirm the presence of nanoporous size scales within the POC polymer networks after selective leaching of the PEGDM porogen. Polymer samples were prepared by leaching in multiple changes of acetone for 48 hours at room temperature. The percent mass loss from the polymer samples was quantified and compared to the original PEGDM weight content of the POC (See FIG. 24). Since polymerized POC is insoluble in water and acetone, PEGDM can be selectively leached from the polymerized POC. As see in FIG. 24, there is a strong correlation between the original PEGDM mass content in the POC polymer and the mass lost after leaching in acetone. Similar results were found for leaching in water for the same period of time (data not shown). After leaching in acetone, samples were critically point dried using a Polaron critical point dryer (Quorum Technologies Ltd., United Kingdom) and sputter coated with gold. Samples were then examined under high resolution SEM (LEO Gemini 1525). FIGS. 25 and 26 are representative SEM images from differently prepared POC polymer samples which contained different percent by wt. content PEGDM during POC polymerization. A control non-porous POC film shown in FIG. 26A shows the absence of pore structures with only presence of surface debris. FIG. 25B shows the polymer surface characteristics with collapsed pore structures after air drying without use of the critical point dryer. FIGS. 25C and 25D show the pore structure characteristics of a 60% and 70% by wt. PEGDM samples. FIG. 26 shows a higher magnification SEM image of a 70% by wt. PEGDM sample showing nano-scale polymer networks and pore structures within the POC material.

### Pore Structure Evaluation Using Mercury Intrusion Porosimetry.

Mercury intrusion porosimetry was assessed using a Micromeritics AutoPore IV 9500 V1.06 to determine pore size distribution, pore volume and density of POC scaffolds. Samples were prepared similarly to the SEM image analysis by first leaching in acetone and then critically point dried prior to mercury intrusion porosimetry. A relationship between applied external pressure and intruded volume of mercury into the pores of the sample material is provided by the Washburn equation [44]:

$$Δ P = \frac{2 σ cos θ}{R}$$

TABLE 3
Summary of mercury intrusion porosimetry.

| | | | | median | |
|---|---|---|---|---|---|
| | | Total | average pore | pore | |
| | Density | pore area | diameter | diameter | Porosity |
| Sample | (g/cm³) | (m²/g) | (nm) | (nm) | (%) |
| POC | 1.3026 | 35.537 | 9.8 | 4.9 | 10.15 |
| FD 75% | 1.1578 | 35.282 | 8.2 | 4.9 | 8.40 |
| PEGDM | | | | | |
| by wt. in POC | | | | | |
| CPD 70% | 0.2336 | 51.582 | 281.7 | 6.0 | 84.83 |
| PEGDM | | | | | |
| by wt. in POC | | | | | |

Preliminary data is summarized in Table 3. A non-porous control POC film was used a means for comparison to the PEGDM leached samples. The POC film had low percent porosity as expected (10.15%) and had average pore diameters of 9.8 nm. In contrast, a critically point dried sample (CPD) which was polymerized using 70% by wt. content PEGDM had a porosity of 84.83% and average and median pore sizes of 281.7 nm. A freeze-dried polymer sample prepared using 75% by wt. PEGDM showed porosity and pore sizes similar to the POC control film, which confirms the polymer pore collapse and polymer chain relaxation which occurs during the freeze drying process.

### Drug Release Evaluation

Since POC polymer exhibits high amounts of autofluorescence, it limits the ability to use fluorescently conjugated antibodies in traditional immunofluorescence detection and other forms of fluorescence detection for quantifying drug release. A 40 KDa neutrally charged biotinylated-dextran molecule (Nanocs Inc., NY, N.Y.) was used in preliminary drug delivery studies to serve as a model growth factor to study encapsulation and release from the POC nanoporous polymer networks. Furthermore, since POC polymer chains contain negatively charged carboxyl groups, the neutrally charged drug analogue can serve as a model growth factor molecule without having charge residue effects which may alter encapsulation and release. Control POC disks (6 mm in diameter) which were polymerized containing 0% by wt. PEGDM and 80% by wt. PEGDM sample disks were drug loaded by soaking in a 25 mg/ml biotin-dextran solution in 1× phosphate buffered saline (PBS) solution at 4° C. for 24 hours. Samples were subsequently freeze-dried and incubated in 2 ml of 1×PBS at 37° C. for in vitro release studies. Sample solutions (2 ml) were taken at corresponding time points and replaced with fresh PBS.

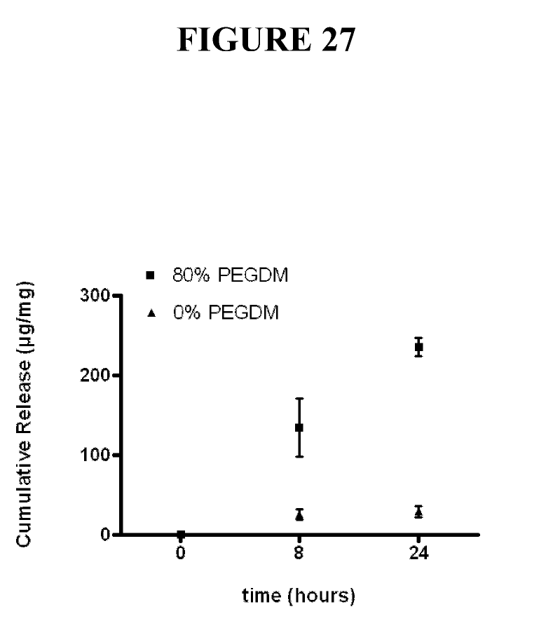

The amount of biotin-dextran released was quantified using a Vitamin H ELISA kit (MD Biosciences Inc., St. Paul, Minn.). The drug release was quantified as total amount of biotin-dextran released per mg of polymer sample. A two-way ANOVA test using Bonferroni post-test analysis was performed on the cumulative drug release data. As shown in FIG. 27, the amount of drug released at 8 and 24 hours for the 80% PEGDM polymer sample was significantly greater than the control 0% PEGDM polymer sample (p<0.001).

### Evaluation of Mechanical Properties.

A preliminary study examined the altered mechanical properties of a POC polymer created using PEGDM as the porogen. Tensile mechanical tests were conducted according to ASTM D412a on an Instron 5544 mechanical tester equipped with 500 N load cell (Instron, Canton, Mass.). Briefly, the dog-bone-shaped sample (26×4×1.5 mm. length×width×thickness) was pulled at a rate of 500 mm/min. Values were converted to stress-strain and a Young's modulus was calculated from the initial sloping region of the stress-strain curve.

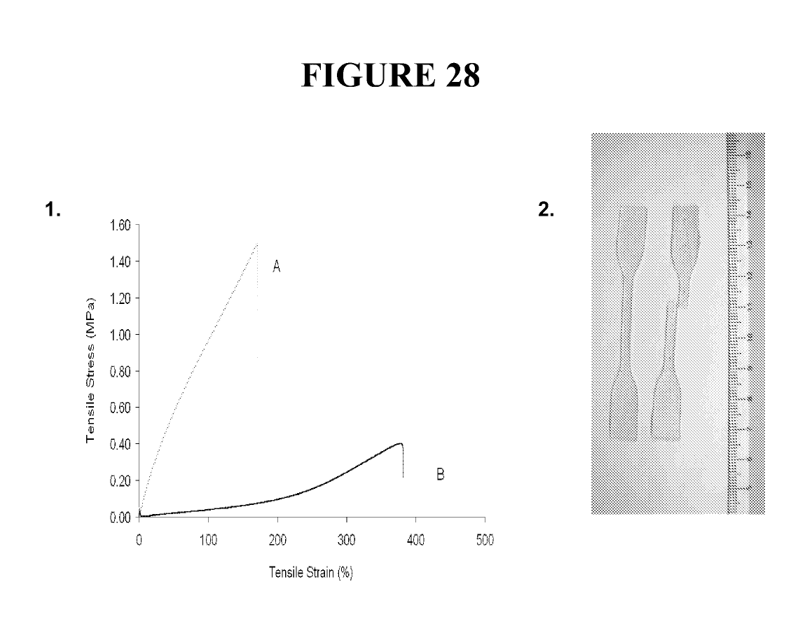

FIG. 28.1 shows representative stress-strain curves for POC polymers created using 0% PEGDM content and 75% by wt. PEGDM content. The 75% PEGDM sample was leached in multiple changes of water for at least 48 hours and then freeze dried prior to mechanical testing. The 0% PEGDM and 75% PEGDM samples had an average elongation at break (%) of 173.69±13.62 and 404.71±17.61 respectively (n=6, SD). These preliminary mechanical tests show that using PEGDM as a porogen during POC polymerization can greatly increase the relative elasticity of POC, which makes it a suitable polymer drug delivery system in conjunction with ePTFE grafts. Furthermore, the 75% by wt. PEGDM polymers showed no permanent plastic deformation after breakage point (see FIG. 28.2).

### Evaluation of Polymer Degradation In Vitro.

Disk-shaped specimens (6 mm in diameter, about 1-1.5 mm thickness) were placed in a tube containing 10 ml phosphate buffer saline (PBS) (pH 7.4) to obtain relative degradation rates among different samples. Specimens were incubated at 37° C. in PBS solution for pre-determined times. After incubation, samples were washed with distilled water and freeze-dried. A total of at least 3 samples were performed for the degradation test at each given time point. Mass loss was calculated by comparing the initial mass (W₀) with the mass measured at a given time point Wₜ as shown in the equation below:

$$Mass loss ( \% ) = \frac{{W}_{0} - {W}_{t}}{{W}_{0}}$$

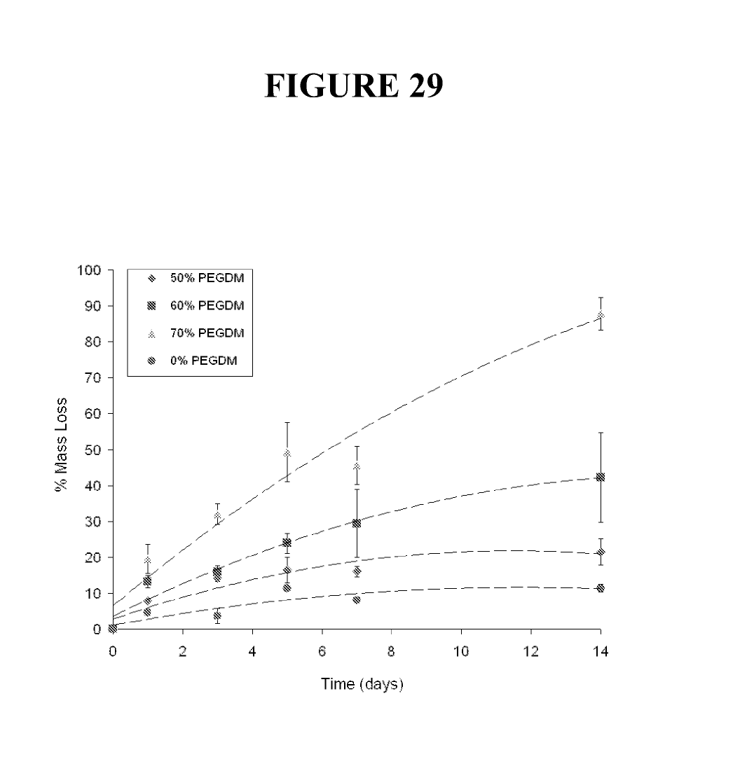

As shown in FIG. 29, by modifying the percent content of PEGDM during POC polymerization, it can alter the degradation rate of the polymer. A 70% by wt. PEGDM sample showed almost complete degradation after two weeks, whereas a 60% and 70% sample had only a % mass loss of 21.44±3.66 and 42.28±12.45 respectively.

### REFERENCES

[1] Langer R, Vacanti I P. Tissue engineering. Science 1993,260:920
[2] Griffith L G. Polymeric biomaterials. Acta Mater. 2000,48:263-277
[3] Guldberg R E. Consideration of mechanical factors. Ann. N. Y. Acad. Sci. 2002;961:312-314.
[4] Wang Y D, Ameer G A, Sheppard B I, Langer R. Nature Biotechnology, 2002;20:602-606.
[5] Greenwald S E, Berry C L. I. Pathol. 2000;190:292-299.
[6] Xue L, Greisler H P I. Vas. Surg. 2003;37:472-480.
[7] Kim B S, Putnam A J, Kulik T I, Mooney D I. Biotechnol. Bioeng. 1998;57:46-54.
[8] Ziegler T, Nerem R M. I. Cell. Biochem. 1994;56:204-209.
[9] K wcon H Y, Yoo M K, Park I K, Kim T H, Lee H C, Lee H S, Oh I S, Akaike T, Cho C S. Biomaterials 2003;24:801-808.
[10] Lee M C, Haut R C. I. Biomech. 1992;25:925-927.
[11] Chiba M, Komatsu K. I. Biomech. 1993;26:561-570.
[12] MisofK, Rapp G, Fmtzi P A. Biophys. I. 1997;72:1376-1381.
[13] Camagey I, Hem-Anderson D, Ranieri I, Schmidt C E. I. Biomed. Mater. Res. Part B: Appl Biomater 2003;65B:171-179.
[14] Consigny P M. Long Term Eff Med Implants 2000;10:79-95.
[15] Bordenave L, Remy-Zolghadri M, Femandez P, Bareille R, Midy D. Endothelium 1999;6:267-275.
[16] Hubbell I A, Massia S P, Desai N P, Drumheller P D. Biotechnology 1991;9:568-572.
[17] Greisler H P, Chatelier R C, Gengenbach T R, Johnson G, Steele I G. Biomater Sci Polymer Ed. 1994;5:531-554.
[18] Bos G W, Poot A A, Beugeling T, van Aken W G, Feijen I. Archieve of Physiology and Biochemistry 1998; 106: 100-115.
[19] Dekker A, Reitsma K. Beugeling T, Feijen I, van Aken W G. Biomaterials 1991;12:130-138. [20] van Wachem P B, Beugeling T, Feijen I, Biomaterials 1985;6:403-408.
[21] Yang J, Bei J Z, Wang S G. Biomaterials, 2002;23:2607-2614.
[22] Yang J, Shi G X, Bei J Z, Wang S G, Cao Y L, Shang Q X, Yang G H, Wang W J. J. Biomed. Mater. Res. 2002,62(3):438.446.
[23] J. Yang, A. R. Webb, G. A. Ameer. Advanced Materials 2004; in press:
[24] F. W. L. Harry R. Allcock,e. New Jersey: Prentice-Hall, Inc. 1990.
[25] L. H. Sperling,e. New York: John Wiley & Sons. 1992.
[26] J. Yang, G. X. Shi, J. Z. Bei, S. G. Wang, Y. L. Cao, Q. X. Shang, G. H. Yang, W. J. Wang. J. Biomed. Mater. Res. 2002; 62: 438.
[27] L. J. Suggs, E. Y. Kao, L. L. Palombo, R. S. Krishnan, M. S. Widmer, A. G. Mikos, in Polymers for Tissue Engineering, (Eds: H. A. H. M. S. Shoichet). Utrecht: VSP. 1998 p. 99.
[28] H. G. Barth,e. New York: Wiley Interscience. 1984.
[29] G. Gouesbet, G. Grehan,e. New York: Plenum Press.1988. [30] B. Chu,e. 2nd ed. New York: Academic Press. 1991.
[31] Y. A. Akpalu, Y. Lin. Journal of Polymer Science: Part B: Polymer Physics 2002; 40:2714. [32] M. S. Lisowski, Q. Liu, J. Cho, J. Runt, F. Yeh, B. S. Hsiao. Macromolecules 2000; 33: 4842.
[33] J. H. v. Zanten, H. G. Monbouquette. Journal of Colloid and Interface Science 1994; 165. [34] M. Siddiq, C. Wu, B. Li. Journal of Applied Polymer Science 1996; 60: 1995.
[35] Y. L. Kim, Y. Liu, R. K. Wali, H. K. Roy, M. J. Goldberg, A. K. Kromine, K. Chen, V. Backman. IEEE J. Sel. Top. Quant. Elect. 2003; 9: 243.
[36] Y. D. Wang, G. A. Ameer, B. J. Sheppard, R. Langer. Nature Biotechnology 2002; 20: 602.
[37] U. W. Gedde,e. 1st ed. New York: Chapman & Hall. 1995.
[38] N. A. Peppas, B. D. Barr, in Hydrogels in Medicine and Pharmacy, (Eds: N. A. Peppas). CRC Press: Boca Raton, Fla. 1988 p. 27.
[39] J. R. Vega, L. M. Gugliotta, V. D. G. Gonzalez, G. R. Meira. Journal of Colloid and Interface Science 2003; 261: 74.
[40] M. Barlett, H. Jiang. Physical Review E. Statistical Physics, Plasmas, Fluids, and Related Interdisciplinary Topics 2002; 65: 031906/1.
[41] E. Szuromi, M. Berka, J. Borbaly. Macromolecules 2000; 33: 3993.
42. Raman, V. I.; Palmese, G. R.; Langmuir 2005, 21, 1539.
43. Reignier, J.; Huneault, M. A.; Polymer 2006, 47, 4703.
44. Yang, J.; Webb, A. R.; Ameer, G. A.; Adv. Mater. 2004, 16, 511.
45. Yang, J.; Webb, A. R.; Pickerill, S. J.; Hageman, G.; Ameer, G. A.; Biomaterials 2006, 27, 1889.
46. Motlagh, D.; Allen, J.; Hoshi, R.; Yang, J.; Lui, K.; Ameer, G.; J. Biomed. Mater. Res. 2007, online Mar. 2, 2007, epub ahead ofprint.
47. Yang, J.; Motlagh, D.; Webb, A. R.; Ameer, G. A.; Tissue Eng. 2005, 11, 1876.
48. Lopez, E R.; Daridon, J L.; Plantier, F.; Boned, C.; Fernandez, J.; Int. J. Thermophysics 2006, 27, 1354.
49. Chandy, T.; Mooradian, D. L.; J Applied Polymer Science, 1998, 70, 2143.
50. Merril E. W.; Saltzman E. W.; J. Am. Soc. Artif. Intern. Organs, 1983, 6, 60.
51. Sinha, V. R. and A. Trehan, J Control Release, 2003, 90, 261.
52. Castellanos, I. J., R. Crespo, and K. Griebenow, J Control Release, 2003. 88, 135.
53. Cleland, J. L., M. F. Powell, and S. J. Shire, Crit Rev Ther Drug Carrier Syst, 1993, 10, 307.
54. Washburn, E. W.; Phys. Rev. 1921, 17, 273.
55. Sperling L H. Introduction to physical polymer science. New York: Wiley; 1992.
56. Wang, Y.; Ameer, G. A.; Sheppard, B.; Langer, R.; Nat. Biotechnol. 2002, 20, 587.
57. Van Krevelen, D W.; Hoftyzer, P J.; Properties of polymers—correlations with chemical structure. Amsterdam: Elsevier publishing company; 1972.
58. J. H. Hildebrand, J. M. Prausnitz and R. L. Scott, Regular and related solutions, New York: Van Nostrand Reinhold Company; 1970.
59. Hansen, Hansen solubility parameters: a user's handbook, Florida: CRC press LLC; 2000.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition comprising an elastomeric citric acid polymer and a plurality of biologically active molecules, wherein said polymer has the formula: 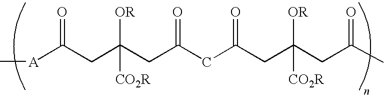
, Where A is a linear aliphatic dihydroxy monomer, C is a linear aliphatic dihydroxy monomer, R is hydrogen or a polymer, and n is an integer greater than 1, and wherein said plurality of biologically active molecules are adsorbed to said nanoporous poly(diol) citrate polymer.

2. The composition of claim 1, wherein A is a linear aliphatic diol comprising between about 2 and about 20 carbons.

3. The composition of claim 1, wherein C is a linear diol comprising between about 2 and about 20 carbons.

4. The composition of claim 1, wherein both A and C are the same linear diol.

5. The composition of claim 1, wherein said polymer is biodegradable.

6. The composition of claim 1, wherein said polymer comprises pores between about 7 and about 15 nanometers.

7. The composition of claim 1, wherein said polymer comprises poly(1,8 octanediol-co-ctric acid).

8. The composition of claim 1, further comprising at least a trace amount of a non-reactive porogen, wherein said non-reactive porogen is impregnated in said polymer.

9. The composition of claim 1, further comprising at least a trace amount of polyethylene glycol dimethyl ether (PEGDM), wherein said PEGDM is impregnated in said polymer.

10. The composition of claim 1, wherein said plurality of biologically active molecules are selected from the group consisting of: proteins, nucleic acids, drugs, pro-drugs, and small molecules.

11. A composition comprising an elastomeric citric acid polymer having the formula: 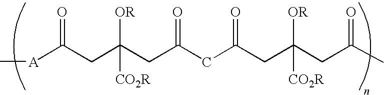
, where A is a linear aliphatic dihydroxy monomer, C is a linear aliphatic dihydroxy monomer, R is hydrogen or a polymer, and n is an integer greater than 1.

12. The composition of claim 11, wherein said polymer comprises pores between about 7 and about 15 nanometers.

13. The composition of claim 11, wherein A and C are each linear aliphatic diol comprising between about 2 and about 20 carbons.

14. The composition of claim 11, wherein said pores have a medium diameter of about 9.5 nanometers.

15. The composition of claim 11, further comprising a plurality of biologically active molecules that are absorbed to said polymer.

16. The composition of claim 11, wherein said plurality of biologically active molecules are selected from the group consisting of: proteins, nucleic acids, drugs, pro-drugs, and small molecules.

17. The composition of claim 11, wherein said polymer is biodegradable.

18. The composition of claim 11, wherein said polymer comprises poly(1,8 octanediol-co-ctric acid).

19. A composition comprising an elastomeric citric acid polymer and at least trace amounts of a non-reactive porogen, wherein said polymer has the formula 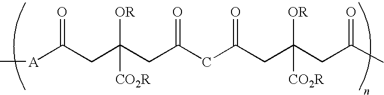
, where A is a linear aliphatic dihydroxy monomer, C is a linear aliphatic dihydroxy monomer, R is hydrogen or a polymer, and n is an integer greater than 1.

20. The composition of claim 19, wherein said nonreactive porogen comprises polyethylene glycol dimethyl ether (PEGDM).
